(19) United States
(12) Patent Application Publication
(10) Pub. No.: US 2025/0037877 A1
(43) Pub. Date: Jan. 30, 2025

(54) PREDICTING ONSET AND PROGRESSION OF NEURODEGENERATIVE DISEASES USING BLOOD TEST DATA AND MACHINE LEARNING MODELS

(71) Applicants: Mor Research Applications Ltd., Ramat Gan (IL); Ariel Scientific Innovations Ltd., Ariel (IL)

(72) Inventors: Amir GLIK, Ramat Gan (IL); Chen HAJAJ, Ariel (IL); Orit REPHAELI, Ariel (IL); Anat GOLDSTEIN, Ariel (IL)

(73) Assignees: Mor Research Applications Ltd., Ramat Gan (IL); Ariel Scientific Innovations Ltd., Ariel (IL)

(21) Appl. No.: 18/910,178

(22) Filed: Oct. 9, 2024

Related U.S. Application Data
(63) Continuation-in-part of application No. PCT/IL2024/050342, filed on Apr. 3, 2024.
(60) Provisional application No. 63/456,555, filed on Apr. 3, 2023.

(51) Int. Cl. G16H 50/30; G16H 10/40; G16H 10/60; G16H 20/17; G16H 50/70

(52) U.S. Cl. CPC G16H 50/30; G16H 10/40; G16H 10/60; G16H 20/17; G16H 50/70

(57) ABSTRACT

Methods and systems for predicting the onset and progression of neurodegenerative diseases using blood test data and machine learning models are disclosed. The method involves receiving blood test values for a target subject over a previous time period, extracting features from the blood test data, and applying a trained predictive model to compute a risk score indicating the probability of developing a neurodegenerative disease. The system includes a processor that executes code to perform the method and can be implemented as a standalone application, cloud-based service, or integrated with healthcare systems. The predictive models are trained using supervised learning on labeled data and can include statistical models or machine learning algorithms. The systems and methods enable early detection of neurodegenerative diseases, personalized risk prediction, and integration with existing healthcare infrastructure. By leveraging blood test data and machine learning techniques, patient outcomes may be improved.

### RELATED APPLICATIONS

This application is a Continuation-in-Part (CIP) of PCT Patent Application No. PCT/IL2024/0500342 having International filing date of Apr. 3, 2024, which claims the benefit of priority of U.S. Provisional Patent Application No. 63/456,555, filed on Apr. 3, 2023. The contents of the above applications are all incorporated by reference as if fully set forth herein in their entirety.

### FIELD AND BACKGROUND OF THE INVENTION

The present disclosure, in some embodiments thereof, relates to predicting onset of neurodegenerative diseases and/or neurodegenerative diseases risk factors, and, more specifically, but not exclusively, to using trained predictive models for predicting onset of neurodegenerative diseases and/or neurodegenerative diseases risk factors.

Neurodegenerative diseases such as, for example, amyotrophic lateral sclerosis, multiple sclerosis, Parkinson's disease, Alzheimer's disease (AD), Huntington's disease, Dementia, multiple system atrophy, prion diseases and/or the like are caused by progressive loss of structure and/or function of neurons, in a process known as neurodegeneration. Such neuronal damage may involve functional degradation and ultimately cell death.

Neurodegeneration can be found in the brain at many different levels of neuronal circuitry, ranging from molecular to systemic. As for today, there is no known way to reverse the progressive degeneration of neurons, neurodegenerative diseases are considered to be incurable.

### SUMMARY OF THE INVENTION

The present invention relates to a computer implemented method for predicting the onset and optionally progression of one or more neurodegenerative diseases using blood test values measured during a certain previous (preceding) time period. More particularly, the invention relates to prediction of the probability of onset of neurodegenerative disease(s) using one or more trained predictive models applied to blood test values of subjects.

Some embodiments of the present invention relate to computer-implemented methods and systems for predicting the onset and progression of neurodegenerative diseases, such as Alzheimer's disease, Parkinson's disease, and Huntington's disease, and/or neurodegenerative diseases risk factors such as: hypertension, hyperlipidemia, diabetes mellitus, depression, insomnia, ischemic heart disease, cerebral small vessel disease, and/or stroke, using blood test data and machine learning models. The method involves the following steps:

Receiving blood test values for a target subject over a previous time period.

Applying a trained predictive model to the blood test values and extracted features to compute a predicted risk score for the onset of a neurodegenerative disease in the target subject during a subsequent time period.

Outputting the predicted risk score.

The system includes a processor that executes code to perform the steps of the method. The system can be implemented as a standalone software application, a cloud-based service, or integrated with an electronic medical record system of a healthcare provider. The trained predictive model is developed using supervised learning techniques on labeled training samples, which include blood test values and corresponding labels indicating the presence or absence of a neurodegenerative disease and/or presence or absence of neurodegenerative diseases risk factors in target subjects. The predictive model can be a statistical model, such as logistic regression, or a machine learning model, such as a random forest or neural network.

The method may further include classifying the target subject into a binary risk category and/or giving a confidence interval result based on comparing the predicted risk score to a threshold value. The predictive model may also be adapted to predict the rate of exacerbation of the neurodegenerative disease using longitudinal data, time-dependent covariates, survival analysis techniques (1230), and/or multi-task learning.

An aspect of some embodiments of the present invention includes a system for predicting onset of neurodegenerative diseases, comprising: at least one processor adapted to execute a code, the code comprising: code instructions to receive values of a plurality of blood test measured for target subject during at least one previous time period, each plurality of blood test values is associated with a respective time stamp, code instructions to receive at least one diagnostic test, and to verify that the at least one diagnostic test indicates non-presence of the neurodegenerative disease and/or neurodegenerative disease risk factor in the target subject during a time interval when the plurality of blood tests were measured, code instructions to apply at least one trained predictive model to compute a predicted risk score for the target subject based on a plurality of features extracted from the plurality of blood test values, the at least one trained predictive model is trained to predict a probability of onset of at least one neurodegenerative disease and/or neurodegenerative disease risk factor in subjects during a subsequent time period based on the plurality of blood test values measured during the at least one previous time period, and code instructions to output the predicted risk score indicative of the probability of onset of the at least one neurodegenerative disease in the target subject during the subsequent time period.

Optionally, further comprising code instructions for excluding the plurality of blood test values from further processing when the at least one diagnostic test indicates presence of the at least one neurodegenerative disease and/or neurodegenerative disease risk factor in the target subject during a time interval when the plurality of blood tests were measured.

Optionally, at least one diagnostic test indicates a state of tau protein and/or amyloid indicative of presence or non-presence of the at least one neurodegenerative disease in the target subject.

Optionally, further comprising code instructions for accessing an electronic health record (EMR) or lab results database of the subject, and verifying at least one of: (1) lack of symptoms correlated with the at least one neurodegenerative disease during the time interval when the plurality of blood tests were measured, or excluding the plurality of blood test values from further processing when the EMR includes symptoms correlated with the at least one neurodegenerative disease, or not having neurodegenerative disease risk factor, and (2) lack of administered medications for treatment of the at least one neurodegenerative disease.

Optionally, the result of the at least one diagnostic test is non-correlated with a stage of the at least one neurodegenerative disease on a predefined clinical scale of a plurality of stages for diagnosing the at least one neurodegenerative disease.

Optionally, the at least one trained predictive model comprises at least one first trained predictive model, wherein the plurality of features comprises a first plurality of features, wherein the predicted risk score comprises a first predicted risk score, wherein the subsequent time period comprises a first subsequent time period, the code further comprises code instructions to apply at least one second trained predictive model to compute a second predicted risk score for the target subject based on a second plurality of features extracted from the plurality of blood test values and/or based on the plurality of blood test values, the at least one second trained predictive model is trained to predict a second probability of onset of at least one risk factor likely to lead to development of the at least one neurodegenerative disease in the target subject during a second subsequent time period prior to the first subsequent time period predicted for onset of the at least one neurodegenerative disease.

Optionally, the at least one second trained predictive model is applied in response to the first predicted risk score being above a threshold, wherein the second predicted risk score indicates probability of onset of the at least one risk factor in view of the first predicted risk score being above the threshold.

Optionally, the at least one risk factor is determined by at least one of the plurality of blood test values are within a target range, wherein the plurality of blood test values are external to the target range during the time interval when the plurality of blood tests were measured.

Optionally, further comprising code for applying an interpretability model to the at least one first trained model to identify at least one first blood test value correlated with the first predicted risk score above a first threshold, and applying the interpretability model to the at least one second trained model to identify at least one second blood test value correlated with the second predicted risk score above a second threshold, and confirming that the at least one first blood test value matches the at least one second blood test value.

Optionally, the at least one second trained predictive model is trained on a training dataset of a plurality of records, wherein a record is for a sample individual, the record including the first blood test values and/or the plurality of extracted features, and a ground truth indicating onset or non-onset of at least one risk factor, and onset or non-onset of the at least one neurodegenerative disease, wherein a plurality of records are for sample individuals with a first time interval of onset of the at least one risk factor followed by a second subsequent time interval with onset of the at least one neurodegenerative disease.

Optionally, in response to the second predicted risk score indicating the at least one risk factor being above a threshold, treating the target subject for preventing onset of the at least one risk factor.

Optionally, further comprising: in response to the predicted risk score being above a threshold, monitoring the EMR or blood results database of the target subject during the subsequent time period to identify at least one of: an administered cognitive evaluation indicative cognitive decline, and/or the at least one diagnostic test indicating presence of the at least one neurodegenerative disease in the target subject, and treating the target subject by administering at least one medication to the target subject effective for delaying or preventing progression of the at least one neurodegenerative disease.

Optionally, the at least one neurodegenerative disease includes Alzheimer's Disease, and at least one medication is selected from drugs against the amyloid protein selected from LEQEMBI, Donanemab, aducanumab, or drugs against the tau protein including Tau-targeted therapies, and drugs aimed at the immune system, selected from TBC-ABb002.

Optionally, further comprising: in response to the predicted risk score being above a threshold, treating the target subject by administering at least one medication known to be effective for preventing onset of the neurodegenerative disease.

Optionally, further comprising in response to the predicted risk score being above a threshold, monitoring the EMR of the target subject during the subsequent time period to identify at least one of: an administered cognitive evaluation indicative of normal cognitive function and/or lack of cognitive decline, and the at least one diagnostic test indicating presence of the at least one neurodegenerative disease in the target subject, and treating the target subject by administering at least one medication to the target subject effective for preventing onset of clinical appearance of the at least one neurodegenerative disease.

Optionally, the at least one neurodegenerative disease includes Alzheimer's Disease, and at least one medication is selected from LEQEMBI, and Donanemab.

Optionally, the at least one trained predictive model generates a respective predicted risk score for each stage of a plurality of sequential stages denoting a disease progression profile defined according to a clinical standard.

Optionally, the at least one trained predicted mode is applied to a temporal sequence of a plurality of sets of the plurality of blood test values, each respective set obtained at a respective historical time interval along the temporal sequence.

Optionally, the at least one trained predictive model is trained on a training dataset of a plurality of records, wherein a record is for a sample individual, wherein the record includes a plurality of sets of the plurality of blood test values and/or the plurality of features for the sample individual, a timestamp indicating date for each set of the plurality of sets, and a ground truth indicating diagnosis of at least one stage of the plurality of stages, and a timestamp indicating date for each stage.

Optionally, further comprising code for creating a training dataset, comprising: accessing a plurality of EMRs for a plurality of sample individuals, analyzing each EMR of the plurality of EMRs for creating a subset of EMRs by: including EMRs of sample individuals diagnosed with Alzheimer's Disease (AD), excluding EMRs of sample individuals diagnosed with neurodegenerative diseases other than AD, or who had other non-AD etiology, correlated with cognitive decline, and including other EMRs of sample individuals not diagnosed with AD and not excluded, as cognitive healthy controls, for each EMR of the subset of EMRs, generating a record including at least one set of the plurality of blood test values, a first timestamp indicating date of the set, and a ground truth indicating AD or cognitive healthy control, and a second timestamp indicating date of diagnosis of AD.

Optionally, EMRs of individuals diagnosed with any one of the following conditions leading to cognitive decline document in the EMR are excluded: Brain Tumors, Creutzfeld-Jacob Disease, Drug & Alcohol-Induced Dementia, Parkinson's disease, Lewy Body dementia, stroke, frototemproal dementia.

Optionally, EMRs of sample individuals diagnosed with AD are identified according to at least one of: a diagnostic field indicating AD, and indication of prescribed pharmaceutical treatments known to be prescribed for AD.

Optionally, further comprising code for analyzing the EMRs of the sample individuals, and classifying each EMR of each sample individual into a classification category selected from: Cognitive healthy controls, AD pateints, Cognitive decline not due to AD, and AD patients with prior non-AD diagnosis which is correlated with cognitive decline.

An aspect of some embodiments of the present invention includes a method for training a predictive model to predict the onset of neurodegenerative diseases, comprising: receiving blood test values for a plurality of subjects over a previous time period, wherein each blood test value is associated with a timestamp and each target subject is associated with a label indicating the presence or absence of a neurodegenerative disease, wherein the label is determined for each subject by receiving at least one diagnostic test, and assigning the label indicating absence when the at least one diagnostic test indicates non-presence of the clinical stage of the neurodegenerative disease in the subject during a time interval when the blood test values were measured, and assigning the label indicating presence when the at least one diagnostic test indicates presence of the neurodegenerative disease, extracting a plurality of features from the blood test values, including at least one of: an aggregation of blood test values over the previous time period, selected from the group consisting of an average value, a maximum value, a minimum value, and a standard deviation, a change pattern in the blood test values over the previous time period, selected from the group consisting of values increasing over time, values decreasing over time, values increasing then decreasing, and significant alternations between increases and decreases, training a predictive model using the extracted features and the labels to predict the onset of neurodegenerative diseases, and outputting the trained predictive model for classification the onset of one or more neurodegenerative diseases of a target.

An aspect of some embodiments of the present invention includes a system for predicting onset of neurodegenerative diseases, comprising: at least one processor adapted to execute a code, the code comprising: code instructions to receive values of a plurality of blood test and optionally basic demographics (Age, Gender) measured for target subject during at least one previous time period, each plurality of blood test values is associated with a respective time stamp, code instructions to apply at least one trained predictive model to compute a predicted risk score for the target subject based on a plurality of features extracted from the plurality of blood test values, the at least one trained predictive model is trained to predict a probability of onset of at least one neurodegenerative disease and/or neurodegenerative diseases risk factor in subjects during a subsequent time period based on the plurality of blood test values measured during the at least one previous time period, and code instructions to output the predicted risk score indicative of the probability of onset of the at least one neurodegenerative disease and/or neurodegenerative diseases risk factor in the target subject during the subsequent time period.

Optionally, further comprising a display device, wherein the code further comprises code instructions to control the display device to display the outputted predicted risk score.

Optionally, the at least one predictive model is trained in at least one supervised training session using a plurality of labeled training samples, each labeled training sample associating values of at least some of the plurality of blood tests measured for a respective test subject during the at least one previous time period with a label indicative of whether or not onset of the at least one neurodegenerative disease and/or neurodegenerative diseases risk factor was detected in the respective test subject.

Optionally, the code further comprises code instructions to classify the probability of onset of the at least one neurodegenerative disease and/or neurodegenerative diseases risk factor in the target subject according to a binary classification based on comparison of the predicted risk score to a certain threshold.

Optionally, the at least one trained predictive model is further adapted and trained to predict a rate of exacerbation of the at least one neurodegenerative disease and/or neurodegenerative diseases risk factor.

Optionally, the at least one trained predictive model is further adapted and trained to classify the target subject into a respective one of subject classes according to a disease progression profile predicted for the target subject.

Optionally, the plurality of blood tests include one or more blood tests selected from a group comprising: absolute basophil count (baso abs), absolute eosinophil count (EOS abs), hemoglobin (Hb), hematocrit (Hct), absolute lymphocyte count (lymp abs), mean corpuscular hemoglobin (MCH), mean corpuscular hemoglobin concentration (MCHC), mean corpuscular volume (MCV), absolute mononucleosis (MONO abs), red blood cell count (RBC), procalcitonin (PCT), platelet count (PLT), white blood cells count (WBC), red cell distribution width (RDW), albumin, calcium, chloride, creatinine, globulin, glucose, magnesium, phosphorus, potassium, protein, sodium, urea, uric acid, aspartate aminotransferase (AST/GOT), gamma-glutamyl transferase (GGT), alanine aminotransferase (ALT/GPT), bilirubin total, thyroid stimulating hormone (TSH), vitamin b12, prothrombin time (PT), Partial thromboplastin time (PTT), bilirubin direct, bilirubin indirect, folic acid, lipid profile, HbA1C, and international normalized ratio (INR).

Optionally, the code further comprises code instructions to rank the plurality of blood tests according to an impact of each of the plurality of blood tests on the performance of the at least one predictive model in computing the predicted risk score.

Optionally, the plurality of blood tests used by the at least one trained predictive model to compute the predicted risk score comprise a subset of highest ranking blood tests.

Optionally, the at least one trained predictive model is further adapted and trained to compute the risk score based on at least one physiological parameter of the target subject in addition to the blood test values, the at least one physiological parameter being selected from a group comprising: blood pressure, electrocardiography (ECG) results, heart rate, weight, body mass index (BMI), and electroencephalogram (EEG) signals.

Optionally, the at least one trained predictive model is further adapted and trained to compute the risk score based on at least one risk factor relating to the target subject in addition to the blood test values, the at least one risk factor being selected from a group comprising: Hypertension, Hyperlipidemia, Ischemic heart disease, cerebral small vessel disease, stroke, Myocardial infarction, depression, insomnia, and Diabetes mellitus.

Optionally, the at least one trained predictive model is further adapted and trained to compute the risk score based on at least one behavioral parameter of the target subject in addition to the blood test values, the at least one behavioral parameter being selected from a group comprising: smoking, alcohol intake, drug use, administered medication, and physical activity.

Optionally, the at least one trained predictive model is further adapted and trained to compute the risk score based on at least one sociodemographic parameter of the target subject in addition to the blood test values, the at least one sociodemographic parameter being selected from a group comprising: gender, race, education, age, weight, and height.

Optionally, the at least one trained predictive model is further adapted and trained to compute the risk score based on at least one medical parameter of the target subject in addition to the blood test values, the at least one medical parameter being selected from a group comprising: a medical condition, a background disease, and administered medication.

Optionally, the plurality of features extracted from the plurality of blood test values include at least one of: an aggregation of values of each blood test over the at least one previous time period, the aggregation selected from a group comprising an average value, a maximum value, a minimum value, and a standard deviation, a change pattern detected in the values of at least one blood test over the at least one previous time period, the change pattern selected from a group comprising values increasing over time, values decreasing over time, values increasing then decreasing, and significant alternations between increases and decreases.

Optionally, the at least one neurodegenerative disease is selected from a group comprising: amyotrophic lateral sclerosis, multiple sclerosis, Parkinson's disease, Lewy body dementia, Progressive supranuclear palsy Alzheimer's disease, Huntington's disease, dementia, mixed dementia, multiple system atrophy, and prion diseases.

Optionally, the at least one previous time period has a duration of one or more years selected from a group comprising 1, 2, 3, 4, 5, 6, 7, 8, 9, and 10 years prior to the subsequent time period.

An aspect of some embodiments of the present invention includes a computer implemented method of predicting onset of neurodegenerative diseases, comprising: using at least one processor for: receiving values of a plurality of blood tests measured for a target subject during at least one previous time period, each plurality of blood test values is associated with a respective time stamp, applying at least one trained predictive model to compute a predicted risk score for the target subject based on a plurality of features extracted from the plurality of blood test values, the at least one trained predictive model is trained to predict a probability of onset of at least one neurodegenerative disease and/or neurodegenerative diseases risk factor in subjects during a subsequent time period based on the plurality of blood test values measured during the at least one previous time period, and outputting the predicted risk score indicative of the probability of onset of the at least one neurodegenerative disease in the target subject during the subsequent time period.

Optionally, the at least one predictive model is trained in at least one supervised training session using a plurality of labeled training samples each associating values of at least some of the plurality of blood tests measured for a respective target subject during the at least one previous time period with a label indicative of whether or not onset of the at least one neurodegenerative disease and/or neurodegenerative diseases risk factor was detected in the respective target subject.

Optionally, the at least one trained predictive model comprises at least one statistical model.

Optionally, the at least one trained predictive model comprises at least one machine learning model.

Optionally, further comprising classifying the probability of onset of the at least one neurodegenerative disease and/or neurodegenerative diseases risk factor in the target subject according to a binary classification based on comparison of the predicted risk score to a certain threshold.

Optionally, in the at least one trained predictive model is further adapted and trained accordingly to predict a rate of exacerbation of the at least one neurodegenerative disease and/or neurodegenerative diseases risk factor.

Optionally, in the at least one trained predictive model is further adapted and trained accordingly to classify the target subject to a respective one of subject classes according to a disease progression profile predicted for the target subject.

Optionally, the plurality of blood tests are selected from a group comprising: absolute basophil count (baso abs), absolute eosinophil count (EOS abs), hemoglobin (Hb), hematocrit (Hct), absolute lymphocyte count (lymp abs), mean corpuscular hemoglobin (MCH), mean corpuscular hemoglobin concentration (MCHC), mean corpuscular volume (MCV), absolute mononucleosis (MONO abs), red blood cell count (RBC), procalcitonin (PCT), platelet count (PLT), white blood cells count (WBC), red cell distribution width (RDW), albumin, calcium, chloride, creatinine, globulin, glucose, magnesium, phosphorus, potassium, protein, sodium, urea, uric acid, aspartate aminotransferase (AST/GOT), gamma-glutamyl transferase (GGT), alanine aminotransferase (ALT/GPT), bilirubin total, bilirubin direct, bilirubin indirect, thyroid stimulating hormone (TSH), vitamin b12, folic acid, lipid profile, HbA1C, prothrombin time (PT), Partial thromboplastin time (PTT), and international normalized ratio (INR).

Optionally, further comprising ranking the plurality of blood tests according to an impact of each of the plurality of blood test to performance of the at least one predictive model in computing the predicted risk score.

Optionally, the plurality of blood tests used by the at least one trained predictive model to compute the predicted risk score comprise a subset of highest ranking blood tests.

Optionally, the at least one trained predictive model is further adapted and trained accordingly to compute the risk score based on at least one physiological parameter of the target subject in addition to the blood test values, the at least one physiological parameter is selected from a group comprising: blood pressure, electrocardiography (ECG) results, heart rate, weight, body mass index (BMI), and electroencephalogram (EEG) signals.

Optionally, the at least one trained predictive model is further adapted and trained accordingly to compute the risk score based on at least one risk factor relating to the target subject in addition to the blood test values, the at least one risk factor is selected from a group comprising: Hypertension, Hyperlipidemia, Ischemic heart disease, cerebral small vessel disease, stroke, Myocardial infarction, depression, insomnia, and Diabetes mellitus.

Optionally, the at least one trained predictive model is further adapted and trained accordingly to compute the risk score based on at least one behavioral parameter of the target subject in addition to the blood test values, the at least one behavioral parameter is selected from a group comprising: smoking, alcohol intake, drug use, administered medication, and physical activity.

Optionally, the at least one trained predictive model is further adapted and trained accordingly to compute the risk score based on at least one sociodemographic parameter of the target subject in addition to the blood test values, the at least one sociodemographic parameter is selected from a group comprising: gender, race, education, age, weight, and height.

Optionally, the at least one trained predictive model is further adapted and trained accordingly to compute the risk score based on at least one medical parameter of the target subject in addition to the blood test values, the at least one medical parameter is selected from a group comprising: a medical condition, and a background disease, and administered medication.

Optionally, the plurality of features extracted from the plurality of blood test values include at least one of: an aggregation of values of each blood test over the at least one previous time period, the aggregation selected from a group comprising an average value, a maximum value, a minimum value, and a standard deviation, a change pattern detected in the values of at least one blood test over the at least one previous time period, the change pattern selected from a group comprising values increasing over time, values decreasing over time, values increasing then decreasing, and significant alternations between increases and decreases.

Optionally, the at least one neurodegenerative disease is selected from a group comprising: amyotrophic lateral sclerosis, multiple sclerosis, Parkinson's disease, Lewy body dementia, Progressive supranuclear palsy Alzheimer's disease, Huntington's disease, dementia, mixed dementia, multiple system atrophy, and prion diseases.

Optionally, the at least one previous time period has a duration of one or more years selected from a group comprising 1, 2, 3, 4, 5, 6, 7, 8, 9, and 10 years prior to the subsequent time period.

Optionally, the subsequent time period has a duration of one or more years selected from a group comprising 1, 2, 3, 4, 5, 6, 7, 8, 9, and 10 years following the at least one previous time period.

An aspect of some embodiments of the present invention includes a computer program product comprising a non-transitory computer readable storage medium retaining program instructions, which program instructions when read by a processor, because the processor to perform the method(s) described herein.

An aspect of some embodiments of the present invention includes a method for training a predictive model to predict the onset of neurodegenerative diseases or neurodegenerative diseases risk factor, comprising: receiving blood test values for a plurality of subjects over a previous time period, wherein each blood test value is associated with a timestamp and each target subject is associated with a label indicating the presence or absence of a neurodegenerative disease, extracting a plurality of features from the blood test values, including at least one of: an aggregation of blood test values over the previous time period, selected from the group consisting of an average value, a maximum value, a minimum value, and a standard deviation, a change pattern in the blood test values over the previous time period, selected from the group consisting of values increasing over time, values decreasing over time, values increasing then decreasing, and significant alternations between increases and decreases, training a predictive model using the extracted features and the labels to predict the onset of neurodegenerative diseases, and outputting the trained predictive model for classification the onset of one or more neurodegenerative diseases or neurodegenerative diseases risk factor of a target.

Optionally, training the predictive model comprises: splitting a dataset comprising the blood test values into a training set, a validation set, and a test set, iteratively updating the parameters of the predictive model using the training set to minimize a loss function, tuning the hyperparameters of the predictive model using the validation set to optimize the performance metric, and assessing the generalization performance of the predictive model using the test set.

It is to be understood that other data may be used for training the predictive model(s) described herein, and/or other data may be fed into the trained predictive model(s) described herein during inference. Examples of other data include blood biomarkers and/or CSF biomarkers such as TAU including in different forms (Ptau 181, pTau 217, Amyloid, synuclein, APOE status, etc. . . . ), and/or image data generated by one or more imaging modalities such as MRI, PET (amyloid, Tau, FDG), CT, EEG, fMRI, and TCD.

Optionally, the machine learning model is selected from the group consisting of a decision tree, a random forest, a gradient boosting machine, a support vector machine, and an artificial neural network.

The above described solutions permit early detection of neurodegenerative diseases and/or neurodegenerative diseases risk factor, allowing for timely intervention and treatment and accurate risk prediction using a combination of blood test data and advanced machine learning techniques. Also scalability and accessibility through various implementation options, including cloud-based services and integration with existing healthcare systems are facilitated and a potential for personalized medicine by predicting individual disease progression rates is allowed.

In summary, the embodiments of the present invention provide a tool for predicting the onset and progression of neurodegenerative diseases and/or neurodegenerative diseases risk factor using readily available blood test data and state-of-the-art machine learning models. This can lead to improved patient outcomes, reduced healthcare costs, and advances in our understanding of these complex diseases.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of a system) for predicting onset of neurodegenerative diseases and/or neurodegenerative diseases risk factor, according to some embodiments of the present invention;

FIG. 2 is a flowchart of a computer-implemented method of predicting onset of neurodegenerative diseases and/or neurodegenerative diseases risk factor, optionally implemented on the system depicted in FIG. 1 according to some embodiments of the present invention;

FIGS. 3, 4, 5 and 6 which are graph charts illustrating importance of each of the selected features for the onset prediction of onset of neurodegenerative diseases and/or neurodegenerative diseases risk factor with respect to several setting of duration of the previous time period during which the blood test values (features) were measured and the subsequent time period, i.e., horizon, during which the onset of the Alzheimer's disease dementia is predicted (estimated) to develop;

FIG. 7 is a table showing the performance results of the trained XGBoost model for several previous time periods for predicting onset of neurodegenerative diseases and/or neurodegenerative diseases risk factor, specifically, one, five and ten years, and several subsequent time periods (horizon), specifically, one, two, three, five, six, seven, eight, nine and ten years, according to some embodiments of the present invention;

FIG. 8 is a flowchart of a computer-implemented method of training a model for predicting onset of neurodegenerative diseases and/or neurodegenerative diseases risk factor, optionally used in the method depicted in FIG. 2, according to some embodiments of the present invention;

FIG. 9 is a graph depicting age distribution for various classes in the population at the end of the study period (in the year 2022) of a new set of experiments, in accordance with some embodiments of the present invention;

FIG. 10 includes graphs depicting gender distribution of cognitive health controls and AD patients participating in the new set of experiments, in accordance with some embodiments of the present invention;

FIG. 11 is a table of characteristics of classes of subjects arranged as part of the new set of experiments, in accordance with some embodiments of the present invention;

FIG. 12 is a table presenting a proportion of AD diagnosed for each train and test dataset as part of the new set of experiments, in accordance with some embodiments of the present invention;

FIG. 13 is a table presenting performance metrics of the selected models for the common biomarkers of the new set of experiments, in accordance with some embodiments of the present invention; and

FIG. 14 is a schematic of an exemplary user interface presenting outcomes of the predictive model(s), in accordance with some embodiments of the present invention.

### DESCRIPTION OF SPECIFIC EMBODIMENTS OF THE INVENTION

The present disclosure, in some embodiments thereof, relates to predicting onset of neurodegenerative diseases or neurodegenerative diseases risk factor, and, more specifically, but not exclusively, to using trained predictive models for predicting onset of neurodegenerative diseases or neurodegenerative diseases risk factor.

As used herein, the phrase “onset of the neurodegenerative disease” refers to development of the neurodegenerative disease in a target subject even when no clinical symptoms are apparent. Onset of the neurodegenerative disease may refer to an early stages of the neurodegenerative disease, when a diagnostic test is positive but clinical symptoms are not discernable. Examples of diagnostic tests and/or stages are described herein.

The terms onset and development may be interchanged.

As used herein, the phrase “risk of onset (or risk of developing) the neurodegenerative disease risk factor” refers to the risk of developing one or more neurodegenerative risk factors in a target subject even when no clinical symptoms of the neurodegenerative risk factor(s) are apparent in the target subject. The prediction of the risk of onset of the neurodegenerative disease risk factor is performed for a time interval during which no clinical symptoms of the target subject are apparent.

An aspect of some embodiments of the present invention relates to systems, methods, computing devices, and/or code instructions (e.g., stored on a data storage device and executable by one or more processors) for using one or more predictive models for computing risk scores indicative of a prediction of onset (i.e., development) of a neurodegenerative disease and/or neurodegenerative diseases risk factor using values of blood tests of a subject that is not diagnosed with the neurodegenerative disease at the time of withdrawal of the blood tests (and/or when the values were measured) and/or is not presenting any clinical symptoms associated with the neurodegenerative disease (e.g., cognitive decline) and/or neurodegenerative diseases risk factor.

Optionally, a diagnostic test that checks for the presence of the neurodegenerative disease even at very early stages when no clinical symptoms are present is negative. Examples and/or additional details of such diagnostic tests are described herein. Alternatively, the diagnostic test may be positive when blood samples are taken, however, no clinical symptoms are present when the blood samples are taken. The biomarker status of the target subject may be unknown at the when blood is withdrawn for testing since the biomarker status is not measured routinely and it may become positive a decade before the clinical disease. I.e., clinically the subject is intact but the biomarker status may be positive without a diagnostic test indicating positive. Using at least one implementation of the predictive model(s) described herein, the risk developing the clinical disease may be predicted. From this prediction, it may be assumed (e.g., estimated) when the subject may become biomarker positive using sophisticated assumptions.

The prediction described herein may use routine blood test values, which may be obtained for reasons other than to be used to predict onset of the neurodegenerative disease or neurodegenerative diseases risk factor. The blood test values may be obtained for other values, for example, as part of routine screening for medical conditions or as annual checkups. As such, the prediction may be performed non-invasively, by accessing previously measured blood test values.

Optionally, the prediction is performed by extracting data from an electronic medical record (EMR) of the subject or from blood results datasets. Alternatively or additionally, the predictive model(s) are trained on records created from EMRs of individuals. The EMR(s) may be analyzed to verify that inclusion criteria are met, and exclusion criteria are not met. The inclusion and exclusion criteria are designed to help ensure that the EMR includes blood test values that are not associated with early onset of the neurodegenerative disease even when no clinical symptoms are present (i.e., subject is not, and/or to validate that the blood test values extracted from the EMR are for prediction of a specific neurodegenerative disease, for example, Alzheimer's Disease, and not for other neurodegenerative diseases that may lead to cognitive decline (e.g., amyotrophic lateral sclerosis, multiple sclerosis, Parkinson's disease, Lewy body dementia, Progressive supranuclear palsy Alzheimer's disease, Huntington's disease, dementia, mixed dementia, multiple system atrophy, and prion diseases, etc. . . . ). Exemplary inclusion and exclusion criteria are described herein.

It is to be understood that the term EMR is not necessarily limiting, and is meant to include any source of data and/or dataset and/or storage device on and/or within which blood test results and/or other test results described herein are saved and/or stored.

Optionally, the prediction is indicative of likelihood of progression of the neurodegenerative disease. For example, in a neurodegenerative disease such as Alzheimer's Disease which has multiple stages, the prediction may indicate when onset of each stage is likely to occur.

Optionally, for the target subject, a prediction of likelihood of onset of one or more risk factors likely to trigger onset of the neurodegenerative disease is predicted. The prediction is performed by one or more predictive models in response to an input of blood tests values, optionally a temporal sequence of multiple sets of blood test values.

It is noted that predicting probability of onset of one or more risk factors which are then likely to trigger onset of the neurodegenerative disease is different than simply using a machine learning model to predict onset of a specific risk factor, since such simple prediction is not performed to take into account the impact of the specific risk factor on onset of the neurodegenerative disease. Moreover, the probability of onset of one or more risk factors which are likely to trigger onset of the neurodegenerative disease is different than computing probability of developing the risk factor alone. It is noted that, the prediction of the probability of onset of one or more risk factors which are likely to trigger onset of the neurodegenerative disease may be different for each individual, based on their blood test values. For example, one individual may be at significant risk of developing diabetes which may trigger Alzheimer's Disease, whereas another individual may be at significant risk of developing hyperlipidemia which may trigger Alzheimer's Disease.

Starting to treat the predicted risk factors as early as possible may prevent onset of the neurodegenerative disease in the future. Balancing risk factors, several decades before the onset of the disease, can prevent the onset of the disease in about 30% of patients. That is why screening for the neurodegenerative disease is so important. Moreover, starting treatment with, for example, the anti-amyloid medication (for example leganemab, donanemab) is indicated and most effective in the beginning of the clinical stage of the disease.

Unless otherwise defined, all technical and/or scientific terms used herein have the same meaning as commonly understood by one of ordinary skill in the art to which embodiments described herein pertain. Although methods and materials similar or equivalent to those described herein can be used in the practice or testing of embodiments described herein, exemplary methods and/or materials are described below. In case of conflict, the patent specification, including definitions, will control. In addition, the materials, methods, and examples are illustrative only and are not intended to be necessarily limiting.

According to some embodiments described herein, there are provided methods, systems, and computer program products for predicting onset of neurodegenerative diseases, for example, amyotrophic lateral sclerosis, multiple sclerosis, Parkinson's disease, Lewy body dementia, Progressive supranuclear palsy Alzheimer's disease, Huntington's disease, dementia, mixed dementia, multiple system atrophy, and prion diseases and/or the like using one or more trained predictive models, for example, statistical models, Machine Learning (ML) models and/or the like.

Aspects of embodiments pertain to non-invasive early detection of risk factors relating to dementia and/or neurodegenerative diseases such as Alzheimer's Disease, to identify risk factors, mitigate the risk factors, e.g., by balancing cardiovascular risk factors, to delay or prevent onset of the disease or in order to give treatment early in disease course.

Embodiments pertain to a method and a computerized platform that is configured to perform the following: receiving a plurality of physiological values for the target individual, wherein each value is associated with a respective time stamp; and computing, with respect to the target individual, based on the plurality of physiological values, an output indicating a confidence of onset of a dementia and/or neurodegenerative disease.

Embodiments pertain to a method and a computerized platform that is configured to predict the risk of developing neurodegenerative disease and/or neurodegenerative diseases risk factor, the confidence of onset of dementia and/or neurodegenerative diseases and/or to determine an estimate about the risk to develop dementia and/or neurodegenerative diseases and/or neurodegenerative diseases risk factor, as well as providing a binary output indicating whether the selected subject is at high risk for Dementia or not and/or neurodegenerative diseases risk factor.

Embodiments pertain to a method and a computerized platform configured to identify one or more risk factors that are associated with a high-level onset confidence value of a dementia and/or neurodegenerative disease onset in the target individual, wherein the high-level onset confidence exceeds a certain predefined threshold.

Embodiments pertain to a method and a computerized platform configured to rank or prioritize the plurality of risk factors to recommend preventive interventions in accordance with the ranked prioritization to reduce the confidence of onset of a dementia and/or neurodegenerative disease in the target individual such to drop below a high-level onset confidence value.

In some embodiments, the system and method may pertain to predicting based on a predictive model, for a target individual, the confidence of onset and/or the risk of developing dementia and/or a neurodegenerative disease and/or neurodegenerative diseases risk factor based, for example, on the blood test results including, for instance, based on a Complete Blood Count (CBC), blood chemistry profile (e.g., electrolytes, enzymes, fats, vitamin level (e.g., B12, etc.), hormonal level (e.g., Thyroid Stimulating Hormone (TSH), etc.), and/or the like, of the target individual. Additional or alternative parameter values such as blood biomarkers and/or CSF biomarkers such as TAU including in different forms (Ptau 181, pTau 217, Amyloid, synuclein, APOE status, etc. . . . ), and/or image data generated by one or more imaging modalities such as MRI, PET (amyloid, Tau, FDG), CT, EEG, fMRI, and TCD, physiological parameter values (e.g., blood pressure, Electrocardiography (ECG), heart rate, weight, Body Mass Index (BMI), etc.), vascular risk factors (e.g., Hypertension, Hyperlipidemia, Ischemic heart disease, Myocardial infarction Diabetes mellitus, etc.), sociodemographic parameters (e.g., age, gender, race, education), and/or behavioral parameters including substance abuse (e.g., smoking, alcohol intake, medication); frequency and type of engagement in physical activity) may be taken into consideration.

Embodiments may also pertain to inclusion of additional variables to improve the predictive ability of the model while evaluating the earliest date on which it can be diagnosed.

Embodiments may also pertain to assessing the rate of exacerbation of the disease and characterizing profiles of subgroups of patients according to the rate of disease progression.

Embodiments pertain to determining the risk for dementia, e.g., by taking a regular blood test and decide, the ML model outputs the risk for the individual developing AD in the future.

In some embodiments, there is provided a method for training a predictive model to predict the onset of neurodegenerative diseases and/or neurodegenerative diseases risk factor using blood test data. The method involves receiving a dataset of blood test values with associated timestamps and labels indicating the presence or absence of neurodegenerative diseases. The method then extracts relevant features from the blood test values, such as aggregations (e.g., average, maximum, minimum, or standard deviation) and change patterns over time (e.g., increasing, decreasing, or alternating trends). These extracted features, along with the corresponding labels, are used to train a predictive model, which is then outputted for classifying the onset of neurodegenerative diseases in target subjects.

Optionally, the dataset is split into a training set, a validation set, and a test set. The training set is used to iteratively update the model's parameters by minimizing a loss function. The validation set is used to tune the model's hyperparameters, optimizing a performance metric such as accuracy or F1 score. Finally, the test set is used to assess the model's generalization performance on unseen data.

Optionally, the types of machine learning models that can be used for the predictive model, including decision trees, random forests, gradient boosting machines, support vector machines, and artificial neural networks.

Development of machine learning models of at least one embodiment has been a testament to the technical challenges and complexities inherent in medical data analysis. A meticulously curated dataset drawn from a huge cohort that took years to assemble, was used for training of at least one embodiment of the machine learning model described herein. This data underwent a rigorous transformation process, passing through layers of sophisticated preprocessing pipelines. Other technical challenges in creating the training dataset include missing value imputation and anomaly detection, and/or developing custom plugins to address the unique challenges presented by diverse medical records.

There were additional technical challenges above obtaining cleaned dataset, which may be considered a significant milestone in itself. The technical challenge is that this process demanded not only technical expertise but also a profound understanding of physiology and pathology. The resulting feature set described with reference to at least one embodiment, born from countless iterations and validations, became the cornerstone of the model's ability to detect patterns with remarkable sensitivity while maintaining a low false positive rate.

At least one embodiment described herein addresses the technical problem of providing a screening tool for neurodegenerative disease (and/or neurodegenerative diseases risk factor), optionally Alzheimer's disease, designed for widespread use for a large population. At least one embodiment described herein improves the technical field of machine learning models and/or improves the field of medicine, by providing a screening test for neurodegenerative disease, and/or neurodegenerative diseases risk factor optionally Alzheimer's disease, designed for widespread use for a large population. At least one embodiment described herein improves upon prior approaches for diagnosing Alzheimer's disease. At least one embodiment described herein provides the practical application of identifying subjects at risk for developing Alzheimer's disease in the future, which may enable taking action to prevent or delay onset, such as early treatment using medications, and/or early treatment to reduce or prevent risk factors of Alzheimer's disease.

At the time of filing of the present disclosure, there are no known screening tools for Alzheimer's disease or dementia that can be used by the general population. There are diagnostic tests for Alzheimer's disease which can be positive up to a decade before the clinical appearance of the disease. These tests include: sophisticated blood tests for tau and amyloid proteins, brain mapping (FDG, amyloid, tau), lumbar puncture to test for amyloid and tau, and brain biopsy. All these tests require the subject to come to the hospital/clinic, and all are invasive. In particular, withdrawing blood for testing for new AD biomarkers (e.g., the diagnostic tests described herein) is invasive, as opposed to interrogating an existent database (e.g., EMR) to obtain previously measured blood test values (which may have been performed for reasons other than screening for neurodegenerative disease and/or risk factors) according to at least one embodiment described herein. In contrast, in at least one embodiment, there is not necessarily a need for the subject to arrive at the hospital for specialized testing aka this screening tool is not invasive. Interrogation of an existing database may be performed to obtain routine blood tests (e.g., blood count and basic chemistry) optionally with other parameters such as the age and/or gender of the subject.

Moreover, existing tests for Alzheimer's disease are diagnostic, that is, if they are positive, the subject is defined as having the disease even if the subject does not yet suffer from the symptoms of the disease. Based on new diagnostic criteria for Alzheimer's disease, the state of a positive diagnostic test result without displaying clinical symptoms is referred to as stage 1 of the disease (out of 6 stages), for example, as described with reference to Revised criteria for diagnosis and staging of Alzheimer's disease: Alzheimer's Association Workgroup by Clifford R. Jack Jr. et al, accessed at https://alz-journals(dot)onlinelibrary(dot)wiley(dot)com/doi/10.1002/alz.13859, incorporated herein by reference in its entirety. At the time of filing of the present disclosure, Alzheimer's disease may be thought of as a creeping disease with several stages, the transition between which is not currently clear. The first stage (i.e., stage 1) according to the new guidelines is when biological markers are positive for Alzheimer's disease but there are no clinical symptoms. The next stage (i.e., stage 2) is a stage in which there are subjective complaints about cognitive decline but in a formal examination there is no cognitive decline. This stage is followed by another stage (i.e., stage 3) in which cognitive decline can be documented by formal means but there is no significant impairment in daily functioning (also referred to as Mild Cognitive Impairment). In following stages, there is increasing amount of cognitive decline in the presence of functional impairment (mild dementia, stage 4), moderate dementia (stage 5), and severe dementia (stage 6).

According to at least one embodiment, a model is trained for identifying the risk of developing Alzheimer's disease in a subject that is not currently experiencing Alzheimer's disease, i.e. before stage 1 (it is important to note that stage 0 is about the genetic disease, i.e. the presence of a gene that will definitely cause the disease and is therefore not related to models described herein which predict risk of developing Alzheimer's disease in subjects without genetic predisposition). That is, the model trained and/or used for inference according to at least one embodiment may be used for screening of Alzheimer's disease, which is different than diagnosing an existing Alzheimer's disease (even when no clinical symptoms are apparent). In some embodiments, a process can identify not only who is at risk of contracting Alzheimer's disease but also when the clinical disease will appear, i.e. whether in a year, two years, three years, etc. Based on the knowledge that the disease lasts an average of 6-8 years, at least one implementation of the model described herein may be used to predict in which stage of the disease the subject is at each time. For example, when the subject is predicted to experience stage 1, when the subject is predicted to experience stage 2, when the subject is predicted to experience stage 3, etc. . . . Predicting future time intervals for different stages of the disease may be referred to herein as predicting the course of the disease.

According to at least one embodiment, a model is trained for predicting (e.g., quantifying) the risk of developing Alzheimer's disease in the future, which is different than diagnosing an existing disease. If a model based on at least one embodiment predicts that the subject is at risk of developing Alzheimer's disease, then early action may be taken. For example, to seek medical advice in order to decide whether to perform the tests to diagnose the disease, to balance risk factors for the disease, to start regular cognitive monitoring once a year, to take medications at an early stage, to prevent or reduce risk of developing risk factors for Alzheimer's disease, etc.

At least one embodiment described herein addresses the technical problem of providing a screening tool for predicting onset of a neurodegenerative diseases risk factor, which is a risk for triggering onset of a neurodegenerative disease such as Alzheimer's disease (and/or other diseases described herein), designed for widespread use for a large population. At least one embodiment described herein improves the technical field of machine learning models and/or improves the field of medicine, by providing a screening test for onset of neurodegenerative diseases risk factor, designed for widespread use for a large population. At least one embodiment described herein improves upon prior approaches for predicting onset of a neurodegenerative disease risk factor. At least one embodiment described herein provides the practical application of identifying subjects at risk for developing a neurodegenerative disease risk factor in the future, which may enable taking action to prevent or delay onset of the neurodegenerative disease risk factor, such as early treatment using medications, and/or early treatment, which may reduce or prevent development of the neurodegenerative disease itself, for example, Alzheimer's disease.

According to at least one embodiment, a model is trained for predicting (e.g., quantifying) the risk of developing one or more neurodegenerative risk factors in the future, which is different than diagnosing an existing neurodegenerative risk factor. If a model based on at least one embodiment predicts that the subject is at risk of developing the neurodegenerative risk factor, then early action may be taken. For example, to seek medical advice in order to decide whether to perform the tests to diagnose the neurodegenerative risk factor and/or neurodegenerative disease, to balance the risk factor, to start regular cognitive monitoring once a year, to take medications at an early stage to prevent or reduce risk of developing the risk factor, etc.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Using predictive model based algorithm may be enable non-invasive, low-cost and highly accurate prediction of future onset of one or more of the neurodegenerative diseases and/or neurodegenerative diseases risk factor which may alert caregivers, for example, doctors to prescribe, and/or recommend enable early preventive measures in attempt to prevent or at least postpone the onset and/or reduce its effects on subjects (patients).

The present invention provides methods, systems and computer program products for predicting onset of neurodegenerative diseases and/or neurodegenerative diseases risk factor in one or more subjects, for example human subjects, using one or more trained predictive models which are trained and learned to predict such onset of the neurodegenerative disease(s) or neurodegenerative diseases risk factor and optionally its progression based on medical history of the target subjects, and more for example based on values of a plurality of blood tests measured for one or more target subjects during a certain previous time period.

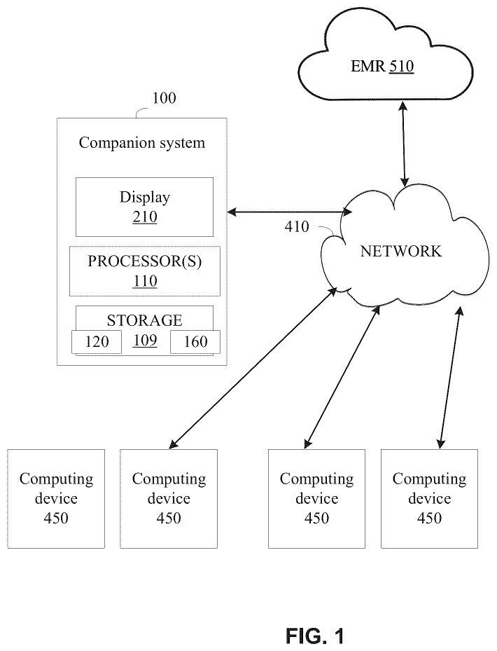

Reference is now made to FIG. 1 which is a schematic illustration of a system (100) for predicting onset of neurodegenerative diseases and/or neurodegenerative diseases risk factor, according to some embodiments of the present invention. The system comprises one or more processors (110) adapted to execute a code (120) and one or more storage units (109) for storing the code and a trained model (160) as described below. The code may include instructions to receive values of a plurality of blood tests measured for target subject(s) during previous time period(s) where each of the blood test values is associated with a respective time stamp. The code further includes instructions to apply trained predictive model (160) to compute a predicted risk score for the target subject(s) based on a plurality of features extracted from the plurality of blood test values. The trained predictive model is trained to predict a probability of onset of at least one neurodegenerative disease and/or neurodegenerative diseases risk factor in subjects during a subsequent time period based on the plurality of blood test values measured during the previous time period(s). This allows the system to output a predicted risk score indicative of the probability of onset of the at least one neurodegenerative disease and/or neurodegenerative diseases risk factor.

The system may comprise or a display device (210) or connected to a client with such a display. This system may instruct a presentation of the outputted predicted risk score on the display.

Implementation of the system (100) can be performed in various ways, depending on the specific requirements and available resources. In one embodiment, the system (100) is implemented as a standalone software application installed on a local computing device, such as a desktop computer, laptop, or server. The standalone software application includes the code and can access the necessary data, such as the blood test values and trained predictive model, from local storage or remote databases (109).

In another embodiment, the system (100) is implemented as a cloud-based service, accessible through a network (410), such as the Internet. The cloud-based service includes the code and necessary data, which are stored and processed on remote servers. Users can access the cloud-based service through a web interface or a dedicated client application installed on their local computing devices (450). This implementation allows for scalability, easy maintenance, and accessibility from various locations.

In yet another embodiment, the system (100) is implemented as a service integrated with or connected to an Electronic Medical Record (EMR) system of a medical institute, such as a hospital, clinic, or research center. System (100) may also be connected to a database of a laboratory service companies, for example, iqvia or insurance companies such as aion, and the like. The service (100) includes the code (120) and can access the necessary data directly from the EMR system (510) or blood sample database. This integration allows for seamless access to patient data, including blood test values and other relevant medical information for training, without the need for manual data entry or transfer. The predicted risk scores can be automatically stored in the EMR system and made available to healthcare professionals and/or subjects themselves for further analysis and decision-making.

Regardless of the implementation method, the system (100) can be configured to ensure data privacy and security, in compliance with relevant regulations and standards, such as the Health Insurance Portability and Accountability Act (HIPAA) or the General Data Protection Regulation (GDPR). This may include implementing access controls, data encryption, and secure communication protocols to protect sensitive patient information.

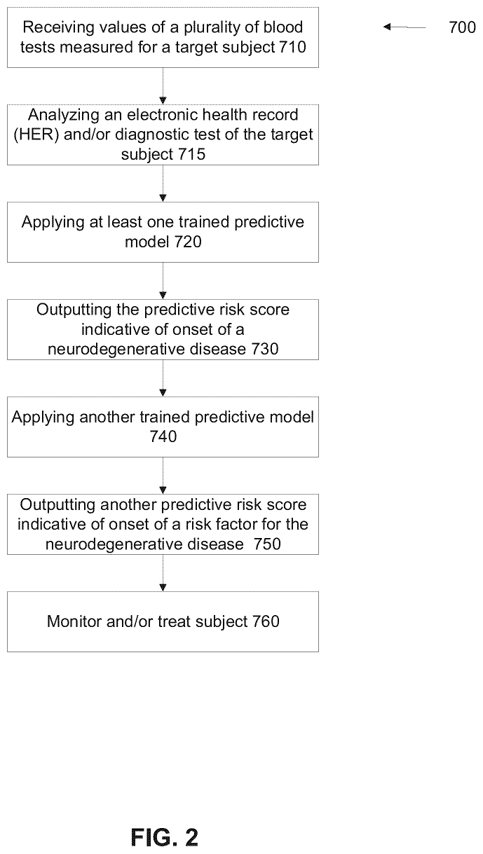

Reference is also made to FIG. 2 which is a flowchart of a computer-implemented method (700) of predicting onset of neurodegenerative diseases and/or neurodegenerative diseases risk factor, optionally implemented on the system (100) depicted in FIG. 1 and described above.

First, as shown at (710), values of a plurality of blood tests measured for target subject(s) during previous time period(s) are received. Each plurality of blood test values is associated with a respective time stamp.

Optionally, as shown at (715), an EMR or other database and/or diagnostic test of the target subject may be accessed and/or analyzed. The EMR stores medical data of the subject, such as the blood tests values, other test results, diagnoses, results of physical examinations, and the like. The EMR may store the diagnostic test.

The access to the EMR and/or diagnostic test may be performed to determine whether the data (i.e., the blood test values and/or other test results fed into the model) of the target subject is eligible for processing, and to proceed with processing the data of the target individual according to the method (i.e., by proceeding with the features of the method), or whether the data of the target subject is non-eligible for processing, and to terminate the method.

It is noted that feature 715 may be implemented, for example, prior to feature 710 and/or at other features of the method.

Optionally, one or more diagnostic tests are automatically accessed. The diagnostic test may be analyzed to verify that the diagnostic test indicates non-presence of the neurodegenerative disease in the target subject during a time interval when the blood tests (obtained as described with reference to 710) were measured. In other words, the diagnostic test indicates that the target subject was diagnosed with the neurodegenerative disease when blood for the blood tests was drawn. The blood test values of the target subject are excluded from further processing when the diagnostic test indicates presence of the neurodegenerative disease in the target subject during a time interval when the blood tests were measured.

Optionally, the result of the diagnostic test is non-correlated with a stage of the neurodegenerative disease on a predefined clinical scale of stages for diagnosing the neurodegenerative disease. For example, even in an early stage in which biomarkers correlated with the neurodegenerative disease are positive but there are no clinical symptoms (e.g., stage 1 in the new AD diagnosis guidelines). Additional exemplary stages are described above and/or with reference to revised criteria for diagnosis and staging of Alzheimer's disease: Alzheimer's Association Workgroup by Clifford R. Jack Jr. et al, accessed at https://alz-journals(dot)onlinelibrary(dot)wiley(dot)com/doi/10.1002/alz.13859, incorporated herein by reference in its entirety

Since as described herein, the model is designed to predict likelihood of onset of the neurodegenerative disease or neurodegenerative diseases risk factor in subjects that do not have the neurodegenerative disease, even in an early stage in which biomarkers correlated with the neurodegenerative disease are positive but there are no clinical symptoms (e.g., stage 1 in the new AD diagnosis guidelines), data of subjects with a positive test for the neurodegenerative disease may be excluded from further processing. Alternatively, a subject is biomarker negative for the neurodegenerative disease (e.g., the diagnostic test(s) described herein is negative) and clinically negative may be included, since the predictive model(s) may predict when the subject will become biomarker or clinically positive.

Examples of diagnostic tests for diagnosing the neurodegenerative disease, in particular AD, include a state of tau protein and/or amyloid indicative of presence or non-presence of the neurodegenerative disease, are described above.

Optionally, the EMR is analyzed to verify one or more, optionally all of the following, for proceeding with processing of the data of the subject:
Lack of clinical symptoms correlated with the neurodegenerative disease during the time interval when the blood tests were measured (i.e., when the blood was drawn). The blood test values are excluded from further processing when the EMR includes presence of clinical symptoms correlated with the neurodegenerative disease.
Lack of administered medications for treatment of the neurodegenerative disease. Presence of prescriptions of medications indicates that the target subject was diagnosed or suspected as having the clinical stage of the neurodegenerative disease.
Lack of risk factors correlated with increased risk of onset of the neurodegenerative disease. The presence or lack of risk factors may be used according to the training of the model, and/or the use of another model, as described herein. For example, in some cases, lack of risk factors is a requirement for further processing, such as when the second model is used to predict onset of risk factors with risk of triggering onset of the neurodegenerative disease and/or neurodegenerative diseases risk factor.

Then, as shown at (720), trained predictive model(s) (160) are applied to compute a predicted risk score for the target subject(s) (140) based on a plurality of features extracted from the plurality of blood test values. The process of applying the trained predictive model(s) to the features and/or blood test values may be also be referred to herein as inference. The trained predictive model (160) is trained to predict a probability of onset of at least one neurodegenerative disease or neurodegenerative diseases risk factor in subjects during a subsequent time period based on the plurality of blood test values measured during the at least one previous time period, for example the blood test exemplified below.

The trained predictive model(s) may be applied to one or more (i.e., combination) of: blood test values, timestamps associated with each set of blood test features, features extracted from the blood test values, non-blood test values (e.g., biopsy results, blood pressure, ECG, EEG), and/or other data which may be extracted from the EMR such as blood biomarkers and/or CSF biomarkers such as TAU including in different forms (Ptau 181, pTau 217, Amyloid, synuclein, APOE status, etc. . . . ), and/or image data generated by one or more imaging modalities such as MRI, PET (amyloid, Tau, FDG), CT, EEG, fMRI, and TCD, age, Gender, and the like. The trained predictive model(s) may be applied to a sequence of values obtained at different time intervals, for example, a temporal sequence of sets of blood tests values, where each set is obtained once a time interval, for example, 7 sets obtained over 7 years (i.e., one test per year).

The predictive model may be a machine learning model.

The predictive model may be implemented using one or more architectures, for example, a binary classifier, a multi-class classifier, a detector, one or more neural networks of various architectures (e.g., convolutional, fully connected, deep, encoder-decoder, recurrent, transformer, graph, combination of multiple architectures), support vector machines (SVM), logistic regression, k-nearest neighbor, decision trees, boosting, random forest, a regressor and the like. Commercial and/or open source package allowing regression, classification, dimensional reduction, supervised, unsupervised, semi-supervised, and/or reinforcement learning may be used. The predictive models may be trained using supervised approaches and/or unsupervised approaches.

This allows, as shown at (730), outputting a predicted risk score indicative of the probability of onset of the at least one neurodegenerative disease and/or neurodegenerative diseases risk factor in the target subject(s) during the subsequent time period.

Optionally, the trained predictive model generates a respective predicted risk score for each stage of multiple sequential stages denoting a disease progression profile defined according to a clinical standard. For example, for the new 6 stage guidelines for diagnosis AD described herein, the trained predictive model may generate a respective likelihood when the target subject is likely to enter stage 1, stage 2, stage 3, etc., until stage 6. Alternatively or additionally, the trained predictive model generates a probability score indicative of likelihood of entering respective stages at sequential times and/or at a common time, for example, 70% for entering stage 1 in 1-3 years, 60% for entering stage 2 in 3-5 years, 55% for entering stage 3 in 5-7 years, etc. Or in another example, 80% for entering stage 1, 65% for entering stage 2, and 47% for entering stage 3, in the next 1-5 years.

Optionally, to generate the outcomes for the sequential stages, the trained predicted mode is applied to a temporal sequence of a set of the blood test values and/or to the set of features extracted from each set of blood test values. Each respective set may have been obtained at a respective historical time interval along the temporal sequence. For example, a respective set of blood tests is obtained once a year, and blood test results are available for the last 7 years, i.e., 7 sets of blood tests over 7 years at 1 year intervals. It is noted that other implementations of the predictive model may be applied to the temporal sequence of blood tests.

Optionally, the trained predictive model is trained on a training dataset of records generated from data of multiple sample individuals. A record may be defined for each sample individual. The record includes multiple sets of blood test values and/or multiples sets of features extracted from the corresponding blood test values. The record may further include a timestamp associated with each set indicating the date on which the blood was drawn and/or the test was performed. The record may include other tests and/or other data described herein. The record may include a ground truth indicating diagnosis of a certain stage of the multiple stages (defined for the neurodegenerative disease), and a timestamp indicating date for each stage. There may be multiple values for different stages associated with different sets of blood tests obtained over time, which may indicate progression of the neurodegenerative disease in the sample individual. For example stage 1 at 2010, stage 2 at 2014, stage 3 at 2012, etc. . . .

Optionally, at (740), another trained predictive model (sometimes referred to herein as a second trained predictive model) may be applied to the blood test values and/or to the extracted features and/or to the timestamps associated with each set of blood test values and/or to other data. The other trained predictive model may be applied to the same blood test values and/or to the same set of extracted features and/or to the same other data, to which the trained model used to predict the score indicating probability of onset of the neurodegenerative model is applied to (also referred to herein as a first trained predictive model). Alternatively or additionally, the other trained predictive model is applied to other blood test values and/or other data. Alternatively or additionally, the other trained predictive model is integrated with the first trained predictive model as a single model, which generates the outcome of the probability of onset of the neurodegenerative model and other outcomes described with reference to feature 740.

The second trained predictive model computes one or more second predicted risk scores for the target subject indicative of (i.e., predicting) a second probability of onset of one or more risk factors likely to lead to development of the neurodegenerative disease (which was predicted by the first predictive model) in the target subject during a second subsequent time period prior to the first subsequent time period predicted for onset of the neurodegenerative disease by the first predictive model. It is noted that the time of prediction of onset of the neurodegenerative disease risk factor and time of predicted onset of the neurodegenerative disease (e.g., AD) may be the same in some subjects.

The risk factors may be risk factors that are diagnosed based on blood tests and/or other tests which are fed into the predictive model(s), for example, hyperlipidemia based on a value above a threshold, of the blood test for one or more of cholesterol, triglycerides, LDL, HDL, etc. . . . In another example, type 2 diabetes may be diagnosed using a value above a threshold, of the blood test for one or more of fasting blood glucose, hemoglobin A1c, glucose tolerance test, etc. . . . In yet another example, high blood pressure may be diagnosed as a blood pressure above a threshold. In yet another example, depression may be diagnosed based on results of a validated clinical evaluation tool, where the results of the tool may be fed into the predictive model(s). Alternatively, the risk factors may be diagnosed based on other parameters which are not necessarily fed into the predictive model(s), for example, clinical symptoms, clinical guidelines, and the like.

Examples of risk factors include Hypertension, Hyperlipidemia, Ischemic heart disease, cerebral small vessel disease, stroke, Myocardial infarction, depression, insomnia, and Diabetes mellitus.

The second predictive model indicates likelihood of onset of the risk factor(s) which are likely to trigger onset of the neurodegenerative disease. A different risk factor may be predicted for different subjects. For example, in one subject there may be a 70% risk of onset of hyperlipidemia in the next 3-5 years, and given the onset of hyperlipidemia, there may be a 50% risk of onset of AD in the 2-5 years after the 3-5 year interval (i.e., in 5-10 years). In another subject there may be a 45% risk of onset of type 2 diabetes mellitus in the next 1-3 years, and given the onset of diabetes, there may be a 60% risk of onset of AD in the next 1-3 years after the initial interval. It is noted that the timeframe of prediction of onset of the neurodegenerative risk factor (e.g., hypertension or others described herein) and timeframe of predicted onset of the neurodegenerative disease (e.g., AD) may overlap or be the same in some subjects.

In some embodiments, predicting probability of onset of one or more risk factors which are then likely to trigger onset of the neurodegenerative disease is different than simply using a machine learning model to predict onset of a specific risk factor, since such simple prediction is not performed to take into account the impact of the specific risk factor on onset of the neurodegenerative disease.

In other embodiments, predicting probability of onset of one or more risk factors is done independently of the neurodegenerative disease.

Optionally, the second trained predictive model is applied in response to the first predicted risk score by the first trained predictive model being above a threshold. The threshold may indicative significant risk of onset, for example, at least about 50%, or 60%, or 70%, or other values. The second predicted risk score indicates probability of onset of the risk factor(s) in view of the first predicted risk score being above the threshold. In other words, give, the risk of onset of the neurodegenerative disease being 70% in the next 5-7 years, the risk of onset of hyperlipidemia which is a risk factor for triggering onset of the neurodegenerative disease is 50% in the next 1-3 years.

The risk factor(s) may be determined by one or more of the blood test values being within a target range. The blood test values (which are fed into the second predictive model) are external to the target range during the time interval when the blood tests were measured and/or when the blood was drawn. For example, blood is drawn indicating a fasting blood glucose level within normal limits. The value of the blood within normal limits is fed into the second model. The second model generates an output indicating that the fasting blood glucose level is predicted to rise to a level indicating diabetes within 2-4 years.

Optionally, an interpretability model is applied to the first trained model to identify at least one first blood test value correlated with the first predicted risk score indicating probability of onset of the neurodegenerative disease (e.g., when the first predicted risk score is above a first threshold). Examples of interpretability models include Local Interpretable Model-agnostic Explanations (LIME), Shapley Additive exPlanations (SHAP), and the like. The interpretability model may be applied to the second trained predictive model to identify at least one second blood test value correlated with the second predicted risk score indicative of probability of onset of one or more risk factors (e.g., when the second predicted risk score is above a second threshold). When the first blood test value matches the second blood test value, a confirmation may be generated indicating that the blood test value predicts onset of the risk factor which is likely to trigger onset of the neurodegenerative disease.

Optionally, the second trained predictive model is trained on a training dataset of records generate from data of multiple sample individuals. A record may be generated from data of a sample individual. The record may include the blood test values and/or extracted features. The record may include and a ground truth indicating onset or non-onset of at least one risk factor. The record may further include an indication of onset or non-onset of the neurodegenerative disease, optionally a stage within the multiple stage definition described herein. The record may further include timestamps indicating the date when the blood tests were obtained, onset of the risk factor, and/or onset of the neurodegenerative disease optionally per stage. Optionally, at least some records are for sample individuals with a first time interval of onset of the risk factor followed by a second subsequent time interval with onset of the neurodegenerative disease, which may indicate that onset of the risk factor triggered onset of the neurodegenerative disease. It is noted that the training described herein may be for generating a single predictive model that combines the first and second predictive models, which outputs risk scores for risk factors and for neurodegenerative disease triggered by the risk factors.

At (750), one or more additional predictive risk scores may be computed for one or more risk factors by the second predictive model(s) described herein.

Optionally, a presentation presenting one or more outcomes of the predictive model(s) described herein is generated. For example, a user interface, optionally a graphical user interface (GUI) is generated and presented on a display.

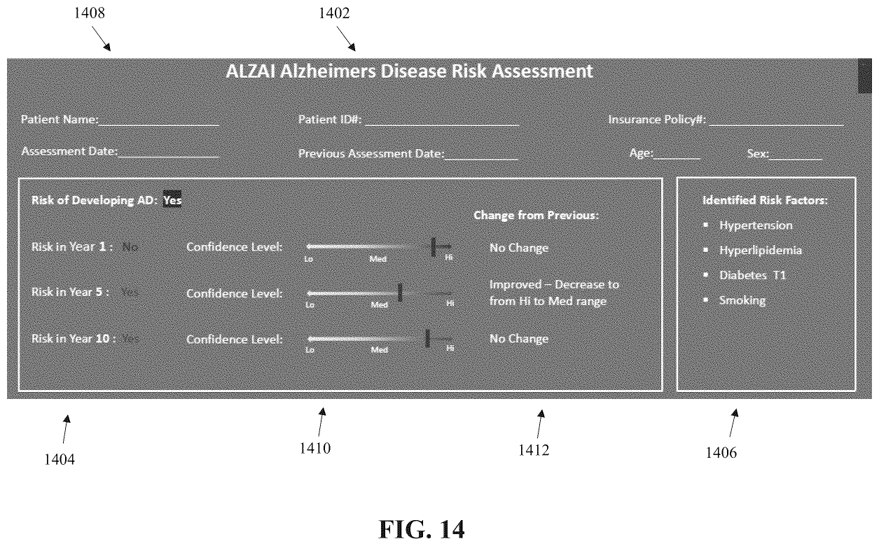

Reference is also made to FIG. 14, which is a schematic of an exemplary user interface (e.g., GUI) 1402 presenting outcomes of the predictive model(s), in accordance with some embodiments of the present invention. GUI 1402 may present the risk scores 1404 outputted by one or more predictive model(s) for onset of the neurodegenerative disease, for example, for different time frames (as shown, risk for years 1, 5, and 10) and/or for different stages of the disease (e.g., disease progression), as described herein. GUI 1402 may present risk scores for onset of one or more risk factors 1406 likely to trigger onset of the neurodegenerative disease, as described herein. The risk scores may be converted into binary values, indicating whether there is a risk or no risk, such as whether the risk score is above a threshold or below a threshold. GUI 1402 may present other data, for example: data associated with the patient 1408, confidence level associated with each risk score 1410, and change from previous prediction 1412.

Referring now back to FIG. 2, at (760), the target subject may be monitored and/or treated accordingly.

The target subject may be monitored and/or treated according to the predicted risk scores for onset of the neurodegenerative disease and/or for onset of one or more risk factors that may trigger the onset of the neurodegenerative disease.

The target subject may be monitored and/or treated according to a combination of the predicted risk scores, and/or whether or not the target subject is displaying clinical symptoms (e.g., cognitive decline), and/or whether or not the target subject is diagnosed with the neurodegenerative disease. Alternatively, the target subject may be monitored and/or treated for the neurodegenerative disease risk factor, for example, for preventing or delaying onset of the neurodegenerative disease risk factor.

Optionally, in response to the predicted risk score indicating onset of the neurodegenerative disease during the subsequent time period meeting a requirement (e.g., being above a threshold), the EMR of the target subject may be monitored prior to and/or during the subsequent time period. The EMR may be monitored to identify an administered cognitive evaluation indicative of cognitive decline. For example, when the predictive model(s) indicates a high risk of developing hypertension (i.e., neurodegenerative risk factor), monitoring for onset of hypertension may be performed by accessing blood pressure values stored in the EMR. In another example, a questionnaire that underwent clinical validation used to monitor cognitive function in a person, where the results of the questionnaire are stored in the EMR of the target subject. The questionnaire may be applied, for example, once per 6 months, once per year, and the like, for tracking changes. Alternatively or additionally, the EMR may be monitored to detect one or more diagnostic tests indicating presence of the neurodegenerative disease in the target subject. Exemplary diagnostic tests are described herein. The diagnostic test may be performed in response to the cognitive evaluation indicating a cognitive decline.

In response to detecting a diagnosis of the neurodegenerative disease and/or cognitive decline, optionally based on the monitoring of the EMR, the target subject may be treated by administering at least one medication effective for delaying or preventing progression of the neurodegenerative disease. When the neurodegenerative disease is Alzheimer's Disease, examples of suitable medications, which may be administered individually or in combination, include:
Drugs against the amyloid protein, for example, LEQEMBI, Donanemab, aducanumab.
Drugs against the tau protein, for example, Tau-targeted therapies.
Drugs aimed at the immune system and/or inflammation, for example, IBC-ABb002.
Other medications according to the state of the subject, which may be determined based on the outcomes of the predictive model(s) and/or other tests, for example, as described with reference to Jeffrey Cummings et al., Alzheimer's disease drug development pipeline: 2024, accessed at https://alz-journals(dot)onlinelibrary(dot)wiley(dot)com/doi/10.1002/trc2.12465, incorporated herein by reference in its entirety.

Alternatively or additionally, in response to the predicted risk score indicating onset of the neurodegenerative disease during the subsequent time period meeting a requirement (e.g., being above the threshold), the EMR of the target subject may be monitored prior to and/or during the subsequent time period. The EMR may be monitored to identify a combination of an administered cognitive evaluation indicative of normal cognitive function and/or lack of cognitive decline and at least one diagnostic test indicating presence of the neurodegenerative disease. In such a case, the target subject may be treated by administering at least one medication effective for preventing onset of clinical appearance of the neurodegenerative disease. For example, when the neurodegenerative disease is Alzheimer's Disease, the medication to prevent onset, is for example, LEQEMBI, and Donanemab.

Alternatively or additionally, in response to the predicted risk score indicating onset of the neurodegenerative disease during the subsequent time period meeting a requirement (e.g., being above the threshold), the target subject is treated by administering at least one medication known to be effective for preventing onset of the neurodegenerative disease. The target subject is treated prior to the subsequent time, when there are no clinical symptoms and even may have no positive biomarkers for the disease, in an effort to prevent the onset of the neurodegenerative disease during the subsequent time period. The target subject may be monitored using the diagnostic test described herein, for determining when the diagnostic test is positive and clinical symptoms have not yet appeared, for preventing onset of the clinical symptoms. There may not necessarily be a need to perform the cognitive evaluation. For example, when the neurodegenerative disease is Alzheimer's Disease, the medication to prevent onset, is for example, LEQEMBI, and Donanemab.

Optionally, in response to the second predicted risk score indicating one or more risk factors meeting a requirement (e.g., being above a threshold), the target subject may be treated for preventing onset of the risk factor(s). For example, medications to reduce lipids may be prescribed to prevent onset of hyperlipidemia, and other medications may be prescribed to prevent onset of diabetes. Starting to treat the predicted risk factors as early as possible may prevent onset of the neurodegenerative disease in the future. Balancing risk factors, several decades before the onset of the disease, can prevent the onset of the disease in about 30% of patients. That is why screening for the neurodegenerative disease or neurodegenerative diseases risk factor is so important.

The blood tests used for training of the model and received as an input as described herein may include various biomarkers and analytes that are relevant to the prediction of neurodegenerative diseases or neurodegenerative diseases risk factor, such as those listed below.

The blood tests which may be obtained from any suitable blood test conducted for the target subject, for example, a plasma test, a serum test, and/or the like may include biomarkers or analytes that are indicative of neurodegenerative diseases.

The blood tests may comprise, for example, absolute basophil count (baso abs), absolute eosinophil count (EOS abs), hemoglobin (Hb), hematocrit (Hct), absolute lymphocyte count (lymp abs), mean corpuscular hemoglobin (MCH), mean corpuscular hemoglobin concentration (MCHC), mean corpuscular volume (MCV), absolute mononucleosis (MONO abs), red blood cell count (RBC), procalcitonin (PCT), platelet count (PLT), white blood cells count (WBC), red cell distribution width (RDW), albumin, calcium, chloride, creatinine, globulin, glucose, magnesium, phosphorus, potassium, protein, sodium, urea, uric acid, aspartate aminotransferase (AST/GOT), gamma-glutamyl transferase (GGT), alanine aminotransferase (ALT/GPT), bilirubin total, thyroid stimulating hormone (TSH), vitamin b12, prothrombin time (PT), Partial thromboplastin time (PTT), international normalized ratio (INR), bilirubin direct, bilirubin indirect, folic acid, lipid profile, HbA1C, and/or the like.

The values of the blood tests may be extracted from medical history records of the target subject which may comprise blood tests values measured during one or more previous time periods, for example, a year, three years, five years, ten years, and/or the like and each of the values of the blood test measurements may be therefore be associated with a respective time stamp indicating the time of taking the respective blood test from which the respective value is obtained. As indicated above, the data may be extracted from any EMR system.

One or more trained predictive models may be applied to the blood tests' values measured for the target subject and/or features derived from such values to compute a predicted risk score based on the plurality of blood test values.

Features extracted from the values of the blood tests may comprise, for example, aggregation of values (scores) of each of one or more blood tests measured over time, for example over the previous time period(s), for example, average value, maximal/minimal values, standard deviation, difference between maximal and minimal values, and/or the like.

The features may further comprise change patterns indicative of change in the blood tests' values change over time. The patterns may be detected by applying transformations on the blood tests' data (values), for example over time-series data comprising values of one or more blood tests measured over time, for example during one or more of the previous time periods, and/or part thereof.

The change patterns may include, for example, footstep graph patterns. These patterns may comprise, for example, values increase over time (also referred to as the “upstairs” pattern), values decrease over time (“downstairs” pattern), an increase of values followed by a decrease (“mountain” pattern), significant alternations between increases and decreases (“fingers” pattern), and/or the like.

Such patterns used as features for training and learning the predictive model(s) may not only enhance its performance but may also simplify its interpretability and/or inference thus gaining further insights into the impact of blood test changes over time on the prediction of one or more of the neurodegenerative diseases.

It should be noted that blood tests and features extracted from these blood tests may be used interchangeably herein after.

The trained predictive model may be trained to predict the probability and/or confidence of onset of one or more of the neurodegenerative diseases in subjects during a certain subsequent time period (horizon), for example, a year, three years, five years, ten years and/or the like based on historical blood test values of target subjects measured during one or more previous time periods.

Specifically, the predictive model(s) may be trained in one or more supervised training sessions using one or more training datasets each comprising a plurality of labeled training samples. Each labeled training sample may comprise values of one or more of historical blood tests measured for a respective one of a plurality of target subjects during one or more of the previous time periods. Each labeled training sample may further associate its historical blood tests values with a label indicative of whether or not onset of one or more of the neurodegenerative diseases was detected (observed) in the respective target subject during one or more subsequent time periods following the previous time period(s).

In other words, during the supervised training session(s), the predictive model(s) may be learned to compute estimated risk score by analyzing historical blood test values measured for each of a plurality of target subjects during one or more of the previous time periods (e.g., 1, 3, 5, 10 years, etc.) coupled with the knowledge of whether or not each of these target subjects was diagnosed with one or more of the neurodegenerative diseases during a subsequent period (e.g., 1, 2, 3, 5, 10 years, etc.) following the (previous) time period(s) during which the blood test values were captured and measured.

One or more of the trained predictive models may comprise, for example, one or more statistical models which, as known in the art, are mathematical representations of one or more real-world phenomena or processes using statistical methods and techniques to analyze and make predictions based on captured and/or measured data. The statistical models, for example, linear regression, logistic regression, time series models, Bayesian models, and/or the like may be therefore used to describe and/or express the relationships between variables, understand the underlying structure of data accordingly and make predictions about future events or outcomes.

In particular, the statistical model(s) may be trained to identify, learn, derive, and/or infer the relationships between the blood test values measured for a target subject and/or features extracted from these blood test values and the probability of the target subject to experience onset of one or more of the neurodegenerative diseases during subsequent period(s) (horizon) and compute a risk score accordingly.

In another example, one or more of the trained predictive models may comprise one or more ML models and/or classifiers, for example, a decision tree, a random forest ensemble, a gradient boost based (XGBoost) classifier, a neural network, a Convolutional Neural Network (CNN), a Deep Learning Neural Network (DNN), and/or the like.

During Their training, the ML models may be applied to the plurality of labeled training samples and, may evolve, adjust, and learned to compute a risk score of a respective subject to exhibit onset of one or more of the neurodegenerative diseases during subsequent period(s) following the (previous) time period during which the historical blood test values were measured.

Training of the ML model(s) may be done, as known in the art, for example, by allocating a plurality of non-overlapping subsets (groups) of the labeled training samples to train, test, and optionally validate the ML model(s). For example, a first subset of the labeled training samples may be allocated to a train dataset used to train the ML model(s), a second subset may be allocated to a test subset used to test the ML model(s), and optionally, a third subset may be allocated to a validation dataset used to validate the ML model(s).

For example, the training process involves at least one supervised training session, where the predictive model (160) is presented with a plurality of labeled training samples. Each labeled training sample consists of two components: input data and a corresponding label. The input data for each labeled training sample includes values of at least some of the plurality of blood tests measured for a respective target subject during the at least one previous time period. These blood test values serve as features that the predictive model (160) learns to associate with the probability of onset of neurodegenerative diseases.

The label for each labeled training sample is indicative of whether or not the onset of the at least one neurodegenerative disease was detected in the respective target subject during a subsequent time period following the at least one previous time period (150) during which the blood test values were measured. The label can be a binary value, such as “onset detected” or “onset not detected,” or it can be a continuous value representing the probability or severity of the onset.

During the supervised training session, the predictive model (160) learns to associate patterns in the input blood test values with the corresponding labels. The model may adjust its internal parameters to minimize the difference between its predicted outputs and the true labels provided in the training samples. This process may allow the model) to learn the relationships between the blood test values and the probability of onset of neurodegenerative diseases. The supervised training session may be performed using various machine learning algorithms, such as decision trees, random forests, support vector machines, or artificial neural networks, depending on the specific implementation of the predictive model. By training the predictive model using a large and diverse set of labeled training samples, the model can learn to accurately predict the probability of onset of neurodegenerative diseases for new, unseen target subjects based on their blood test values.

It should be noted that the model maybe any statistical model as a linear regression model were the probability of onset of neurodegenerative diseases is estimated as a linear combination of the blood test values and extracted features. Alternatively, the model maybe a logistic regression model that estimates the probability of the output variable belonging to a specific category based on a linear combination of the input features or a time series model that receives the data as time series, i.e., sequences of blood test values measured over time. A series model, such as autoregressive model or moving average model, may capture the temporal dependencies in the data and can be used to predict future values of the time series or the probability of an event occurring at a specific time point.

Optionally, the probability of onset of one or more of the neurodegenerative diseases in the target subject may be classified, optionally by one or more of the trained predictive models, according to a binary classification, for example, YES or NO. The binary classification, for example, Yes and NO classes may be determined, for example, based on comparison of the predicted risk score, computed by the trained predictive model(s), to a certain threshold.

As such, in case the predicted risk score exceeds the certain threshold, the target subject may be classified as having a high probability of developing the neurodegenerative disease(s), and vice versa, in case the predicted risk score does not exceed the certain threshold, the target subject may be classified as having a low probability of developing the neurodegenerative disease(s).

Optionally, the probability of onset of the neurodegenerative disease in the target subject is done according to a binary classification where the output variable can take on one of two possible values, typically represented as 0 and 1, or “negative” and “positive”. In the context of the method, the binary classification assigns the target subject to one of two classes based on their predicted risk score:

Class 0 (or “negative”): The target subject is predicted to have a low probability of onset of the at least one neurodegenerative disease.

Class 1 (or “positive”): The target subject is predicted to have a high probability of onset of the at least one neurodegenerative disease.

The binary classification is performed by comparing the predicted risk score to a certain threshold. When the predicted risk score is below the threshold, the target subject is assigned to Class 0. Conversely, if the predicted risk score is above the threshold, the target subject is assigned to Class 1.

The choice of the threshold can be determined based on various factors, such as: the desired balance between sensitivity and specificity, the prevalence of the neurodegenerative disease in a respective population of the target, and/or estimated costs and benefits associated with correct and incorrect classifications (i.e., failing to identify a target subject who will develop the neurodegenerative disease is high, a lower threshold may be more appropriate to minimize the number of false negatives). The binary classification can be performed using various methods, such as thresholding the output of a regression model, using a logistic regression model, using a decision tree or random forest model and/or the like. The binary classification provides a simple and interpretable way to categorize target subjects based on their predicted risk of developing a neurodegenerative disease. This information can be used to guide further diagnostic tests, interventions, or lifestyle changes for target subjects identified as high-risk.

The certain threshold may be set, defined, and/or predefined according to one or more performance metrics and/or parameters, for example, accuracy, precision, recall, F1 factor, and/or the like. For example, a high threshold may be set to achieve high accuracy. However, such high threshold may obviously yield an increased number of false negatives meaning that subjects scored with relatively high risk scores which do not exceed the high threshold may be classified as NO and missed while in practice they may be in potential high risk of developing one or more of the neurodegenerative diseases. In the case of predicting onset of the neurodegenerative diseases, it may be desired to reduce the threshold to ensure no false negatives, typically at the expense of increased false positives.

Optionally, the plurality of blood tests and/or the features extracted from these blood test may be ranked and/or prioritized according to their impact, importance, and/or contribution to performance of the predictive model(s) in predicting the risk score, for example, accuracy, reliability, consistency, and/or the like. This means that during the training, the contribution of each blood test and/or feature may to performance of the trained predictive model(s) may be evaluated and ranked accordingly compared to the other blood tests, for example, blood tests determined to have high contribution may be ranked with higher ranking scores while blood tests determined to have low contribution may be ranked with lower ranking scores. The ranking and/or prioritization of the blood test values and/or features and/or other input data may be obtained by applying an interpretability model to the predictive model(s), for example, Local Interpretable Model-agnostic Explanations (LIME), Shapley Additive exPlanations (SHAP).

Moreover, based on their ranking, the values of only a subset of the plurality of blood tests and/or features may be used to train the predictive model(s) and thus used by the predictive model(s) to compute the estimated risk factor, for example a subset of highest ranking blood tests. For example, assuming that it is determined that values of a subset of eight highest ranking blood tests (and/or extracted features) have a major contribution to the performance of the trained predictive model(s) while using the values of additional blood tests (and/or extracted features) has almost no or only a negligible impact on the performance. In such case, the trained predictive model(s) may be applied to the values of only a subset of blood tests comprising the eight highest ranking blood tests to compute the predicted risk score. Using only the most important blood tests may reduce complexity of the predictive model(s), may improve prediction accuracy, reduce processing resource consumption (e.g., processing resources, storage resources, etc.), reduce processing time, and/or the like.

The predicted risk score computed for the target subject by the trained predictive model(s) may be output to indicate of the probability of onset of one or more of the neurodegenerative diseases in the respective target subject during one or more of the subsequent time periods. The predicted risk score may be output in one or more forms, formats, and/or representations, for example, a numerical value, a binary value, and/or the like expressing, for example, risk and/or probability of the respective target subject to experience onset of one or more of the neurodegenerative diseases during the subsequent time period(s).

For example, one or more care givers, for example, a physician, a therapist, and/or the like may prescribe one or more treatments, medication, an activities, and/or the like to each of one or more target subjects according to his predicted risk score. In another example, the predicted risk score computed for one or more target subjects may be output to one or more automated medical, health and/or treatment systems, for example, a monitoring system, a medical diagnosis system, and/or the like which may be adapted to monitor, diagnose, and/or treat the respective subject according to his computed predicted risk score.

Optionally, one or more of the trained predictive models may be further adapted and trained accordingly to predict a rate of exacerbation of one or more of the neurodegenerative diseases in one or more of the target subjects. For example, the predictive model(s) may be trained to predict how fast (rapid) the neurodegenerative disease(s) will progress in the target subject. In another example, the predictive model(s) may be trained to predict one or more effects of the neurodegenerative disease(s) in the target subject, for example, degradation of one or more abilities, for example, cognitive skill, memory, inference, association, inter-person, communication, reading, and/or the like.

Optionally, one or more of the trained predictive models may be further adapted and trained accordingly to classify each of one or more target subjects to a respective one of a plurality of subject classes according to a disease progression profile predicted for the respective target subject. This means that the trained predictive model(s) may predict a progression profile of one or more of the neurodegenerative diseases in one or more target subjects and assign the respective subject to a specific class based on his disease progression profile. The disease progression profile may comprise one or more parameters, such as, for example, a predicted effects of a neurodegenerative disease, rapidness of disease progress, and/or the like.

Optionally, one or more of the trained predictive models may be further adapted and trained accordingly to compute the risk score based on one or more physiological parameters of the target subject in addition to the blood test values and/or their extracted features. The physiological parameters, for example, blood pressure, Electrocardiography (ECG) results, heart rate, weight, Body Mass Index (BMI), Electroencephalogram (EEG) signals, and/or the like may be measured, captured, extracted, and/or the like using one or more methods, techniques, and/or procedures, as known in the art.

Optionally, one or more of the trained predictive models may be further adapted and trained accordingly to compute the risk score based on one or more vascular risk factors relating to the target subject in addition to the blood test values and/or their extracted features. The vascular risk factors, for example, Hypertension, Hyperlipidemia, Ischemic heart disease, Myocardial infarction Diabetes mellitus, and/or the like may be measured, captured, extracted, and/or the like using one or more methods, techniques, and/or procedures, as known in the art.

Optionally, one or more of the trained predictive models may be further adapted and trained accordingly to compute the risk score based on one or more behavioral parameters of the target subject in addition to the blood test values and/or their extracted features. The behavioral parameters may include, for example, smoking, alcohol intake, drug use, administered medication, physical activity, and/or the like which are known to have at least some impact and/or effect on onset, and/or progression of one or more of the neurodegenerative diseases.

Optionally, one or more of the trained predictive models may be further adapted and trained accordingly to compute the risk score based on one or more sociodemographic parameters of the target subject in addition to the blood test values and/or their extracted features. The behavioral parameters may include, for example, gender, race, education, age, weight, height, and/or the like which are known to have at least some impact and/or effect on onset, and/or progression of one or more of the neurodegenerative diseases.

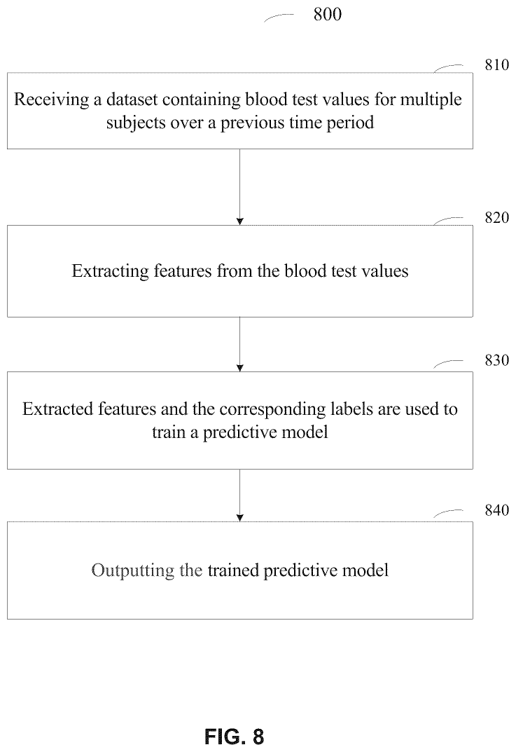

Reference is now made to FIG. 8 which is a flowchart of a method for training a predictive model to predict the onset of neurodegenerative diseases and/or neurodegenerative diseases risk factor, such as the model used by the above described systems and methods.

As shown at 810, the method begins by receiving a dataset containing blood test values for multiple subjects over a previous time period. Each blood test value is associated with a timestamp, indicating when the test was performed, and each target subject is labeled as either having or not having a neurodegenerative disease.

Next, as shown at 820, the method involves extracting features from the blood test values. These features can include aggregations of the blood test values over the previous time period, such as the average value, maximum value, minimum value, or standard deviation. Additionally, the features can include change patterns in the blood test values over time, such as values increasing, decreasing, increasing then decreasing, or showing significant alternations between increases and decreases.

Next, as shown at 830, the extracted features and the corresponding labels are used to train a predictive model to predict the onset of neurodegenerative diseases.

As shown at 840, the trained predictive model is then outputted for classifying the onset of one or more neurodegenerative diseases in a target subject, for instance as described above.

The feature of the method described with reference to FIG. 8 may be applied for training other implementation of the predictive model, for example, as described with reference to FIG. 2. Some exemplary training features are now described.

A training dataset for training at least one predictive model described herein, for example, with reference to FIG. 2, may be created. The training dataset may be created by accessing multiple EMRs storing data of multiple sample individuals and/or lab tests databases. Relevant data, including values of blood tests, may be extracted from the EMRs and/or lab tests databases. Each EMR (i.e., the data extracted from each EMR) and/or lab tests databases may be analyzed, for creating a subset of EMRs and/or lab tests databases, by including or excluding certain EMRs and/or or lab tests databases, as follows:
EMRs and/or or lab tests databases (i.e., the data extracted from each EMR and/or lab tests databases) of sample individuals diagnosed with a specific neurodegenerative disease, for example, Alzheimer's Disease, are included in the subset. EMRs and/or lab tests databases of sample individuals diagnosed with AD may be identified according to at least one of: a diagnostic field indicating AD, and indication of prescribed pharmaceutical treatments known to be prescribed for AD.
EMRs and/or lab tests databases of sample individuals diagnosed with neurodegenerative diseases other than AD, or who had other non-AD etiology, correlated with cognitive decline, are excluded from the subset. It is noted that in some implementations, sample individuals diagnosed with neurodegenerative diseases other than AD, or who had other non-AD etiology, correlated with cognitive decline, may be included, optionally selectively, such as for generative other predictive model(s) for prediction of onset of non-AD pathologies.
EMRs and/or lab tests databases of individuals diagnosed with any one of the following conditions leading to cognitive decline documented in the EMR and/or lab tests databases are excluded: Brain Tumors, Creutzfeld-Jacob Disease, Drug & Alcohol-Induced Dementia, Parkinson's disease, Lewy Body dementia, stroke, frototemproal dementia, amyotrophic lateral sclerosis, multiple sclerosis, Progressive supranuclear palsy, Huntington's disease, dementia, mixed dementia, multiple system atrophy, and prion diseases.
EMRs and/or lab tests databases of sample individuals not diagnosed with AD and not excluded for other reasons, may be included as cognitive healthy controls, optionally labelled with a ground truth label indicating control.

Optionally, each EMR is classified a classification category. The classification categories may be used for determining which EMRs to include in the subset and which EMRs to exclude from the subset. Exemplary classification categories include: Cognitive healthy controls, AD pateints, Cognitive decline not due to AD, and AD patients with prior non-AD diagnosis which is correlated with cognitive decline.

A record is generated for each EMR of the subset of EMRs. Each record includes at least one set of blood test values, optionally multiple temporally spaced sets which may form a sequence over time (e.g., one set per year over several years). The record may further include a first timestamp for each set indicating date of the set, such as date when the blood was withdrawn. The record may include a ground truth indicating whether the sample individual is diagnosed with AD or is a cognitive healthy control. The record may include a second timestamp indicating date of diagnosis of AD. For subjects diagnosed with AD, the record may include the stage of the disease, and optionally a timestamp for each stage indicating the date of diagnosis of the stage.

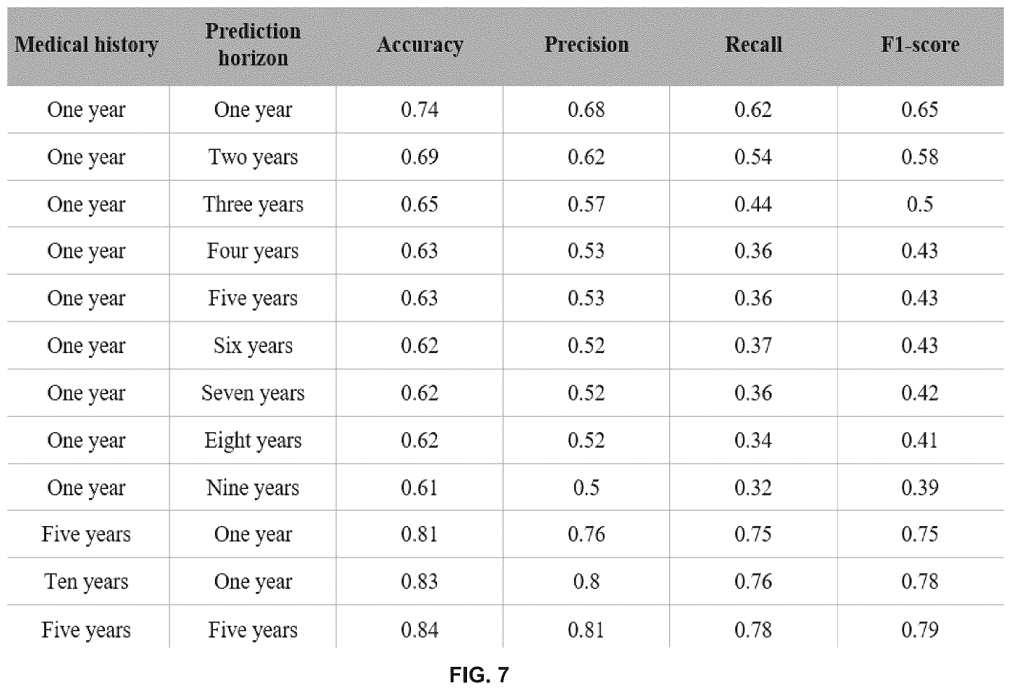

It is to be understood that the process of selecting the EMR(s) for creation of the training dataset may be used to select which EMR is suitable for being processed for feeding into the predictive model(s), for example, using the process described with reference to FIG. 7. For example, the EMR of the target subject may be analyzed to validate that the exclusion criteria described herein are not met and the inclusion criteria described herein are met.

Optionally, the dataset is split into three subsets: a training set, a validation set, and a test set. The training set is used to iteratively update the parameters of the predictive model, minimizing a loss function that quantifies the difference between the predicted and actual labels. The validation set is used to tune the hyperparameters of the predictive model, such as the learning rate, regularization strength, or number of hidden layers in a neural network. Hyperparameters are adjusted to optimize a performance metric, such as accuracy, precision, recall, or F1 score. Finally, the test set is used to assess the generalization performance of the predictive model. This involves evaluating the model's ability to accurately predict the onset of neurodegenerative diseases on data that was not used during the training or validation process.

Optionally, the predictive model is based on one or more various machine learning algorithms, such as decision trees, random forests, gradient boosting machines, support vector machines, or artificial neural networks.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a not necessarily limiting fashion.

Some exemplary experiments were conducted to demonstrate performance of trained predictive models in predicting onset of neurodegenerative diseases and their improvement over existing and/or traditional prediction methods.

Several predictive models (classification models) were evaluated, specifically, a decision tree, a random forest ensemble comprising a plurality of decision trees, and an XGBoost ML model.

The experiments were based on data collected based on retrospectively examination of variables of several thousand control subjects and several thousand diagnosed demented subjects (patients). The demented population was defined according to the National Institute on Aging (NIA) 2011 guidelines for the diagnosis Alzheimer's disease dementia.

For both the demented population (subjects) and the control subjects, the values of the historical blood tests (exams) reflect blood tests taken during a ten or less years previous time period prior to diagnosis. Blood tests with the highest risk for dementia, based on clinical knowledge, were included in the model.

The following datasets and results relate to an experiment in which an XGBoost predictive Model was trained using values of blood test taken from 14,249 control subjects and 9,232 diagnosed subjects (patients). Specifically, the XGBoost Model was adapted and trained to apply binary classification indicating whether a respective subject is estimated to develop Dementia or not.

The blood tests which were evaluated and analyzed were based on complete blood count and Chemistry panel (not all subjects had all data points), and include Baso Abs, EOS Abs, HB, HCT, Lymp Abs, MCH, MCHC, MCV, MONO abs, RBC, PCT, PLT, WBC, RDW, ALBUMIN, CALCIUM, CHLORIDE, CREATININE, GLOBULIN, GLUCOSE, MAGNESIUM, PHOSPHORUS, POTASSIUM, PROTEIN, SODIUM, UREA, URIC ACID, GOT (AST), GGT, GPT (ALT), BILIRUBIN TOTAL, TSH, B12, PT, PTT, and INR.

Based on analysis of the blood tests and their importance, i.e., contribution and/or impact on performance of the XGBoost predictive Model, a subset of the highest ranking blood tests was selected as features for training and using the XGBoost predictive model. The subset comprises Calcium, Vitamin B12, Folic acid, RBC, Albumin, Creatinine, Globulin, Glucose, and WBC. It is noted that in the following experiment described below, a different set of blood test were used.

A probability threshold of 0.5 is defined for the binary classification made by the XGBoost model for differentiating between subjects predicted to be diagnosed with onset of Alzheimer's disease dementia and subjects which are not predicted to experience such onset.

The results listed in the table provided in FIG. 7, showing the performance results of the trained XGBoost model for predicting onset of neurodegenerative diseases and/or neurodegenerative diseases risk factor several previous time periods, specifically, one, five and ten years, and several subsequent time periods (horizon), specifically, one, two, three, five, six, seven, eight, nine and ten years.

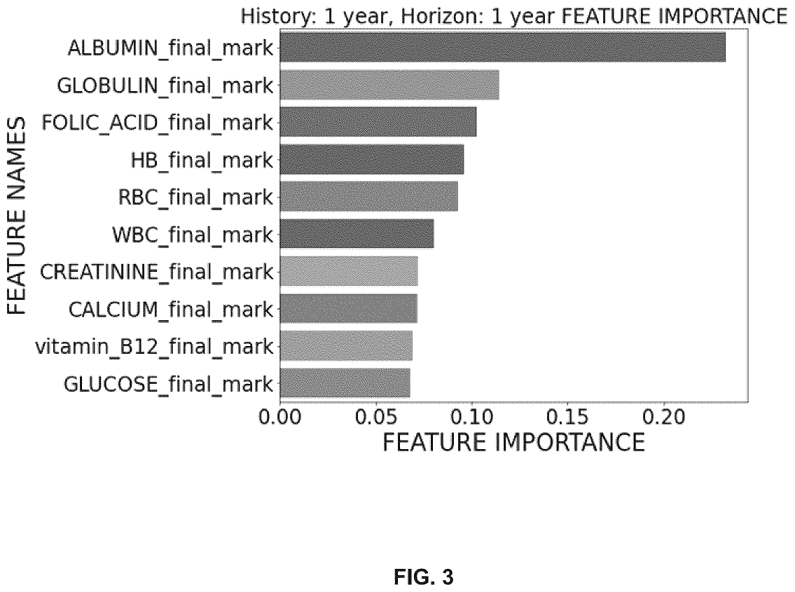

Following are reference to FIGS. 3 to 6 which are graph charts illustrating importance of each of the selected features for the onset prediction with respect to several setting of duration of the previous time period during which the blood test values (features) were measured and the subsequent time period, i.e., horizon, during which the onset of the Alzheimer's disease dementia is predicted (estimated) to develop.

As may be seen in the graph charts, for example from the comparative graph in FIG. 3, some of the blood test values (features) may have larger contribution than others on performance of the trained predictive model meaning their importance to the and may be therefore ranked higher based on their importance.

Moreover, as evident form the graph charts, the importance and thus the ranking of the blood test values (features) may depend on the duration (length) of the previous time period during which the blood test values are measured and/or the duration of the subsequent time period (horizon) for which the prediction of onset is made.

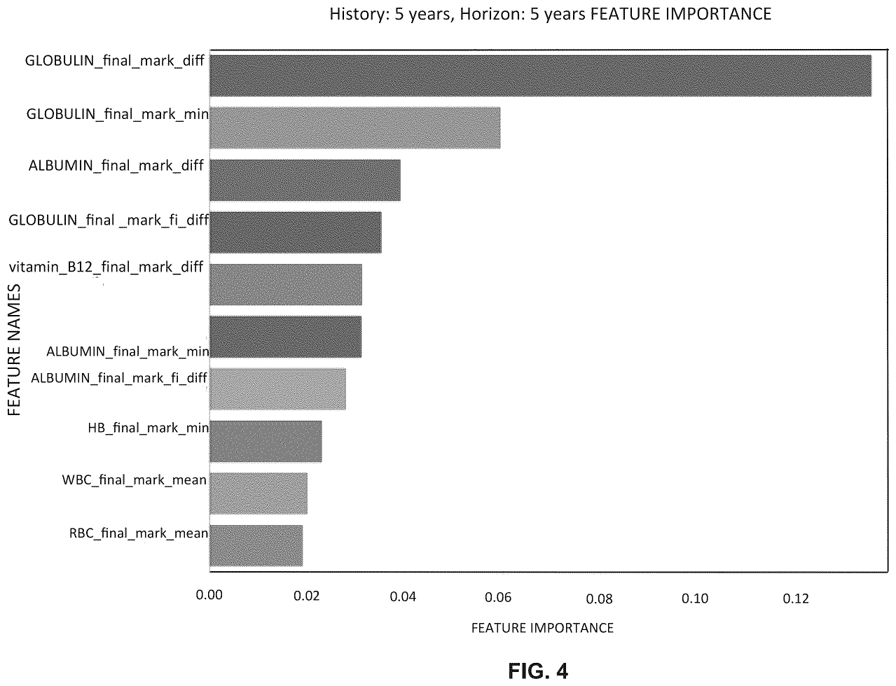

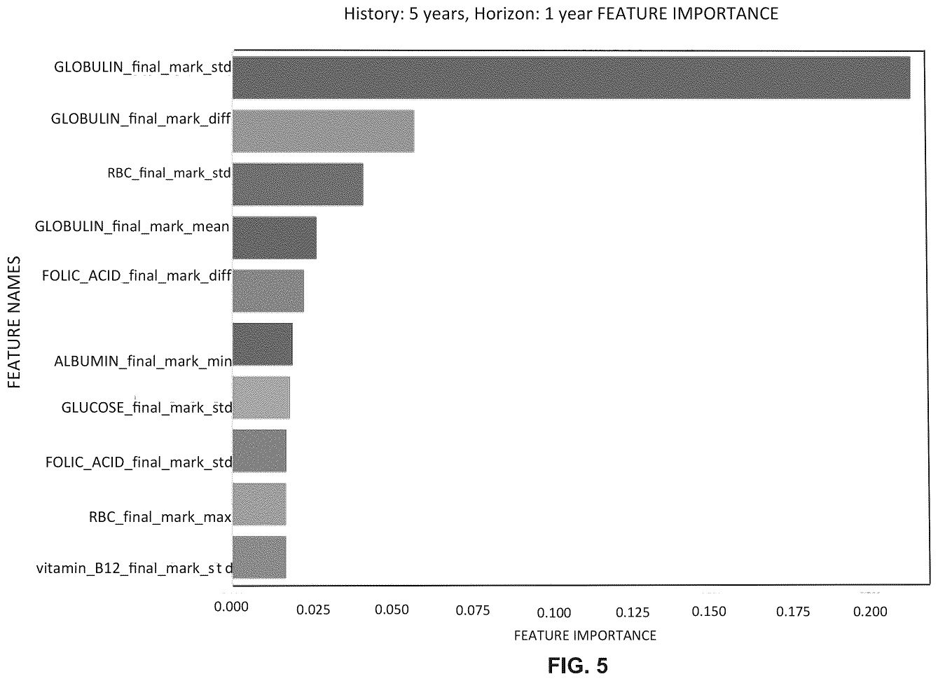

For example, in the graph chart of FIG. 4, showing a one year horizon (subsequent time period) prediction based on a one year previous time period (history), Albumin final mark (value) is the highest ranking feature and Glucose final mark (value) is the lowest ranking one. However, in the graph chart of FIG. 5, showing a one year horizon (subsequent time period) prediction based on a five years previous time period (history), Globulin standard final mark (value) is the highest ranking feature and B12 vitamin standard final mark (value) is the lowest ranking one.

For each blood test several features (variables) were created and/or extracted, for example, average score for all years, maximal score for all years, minimal score for all years, standard deviation of the score for all years, difference between the maximal score and the minimal score for all years, difference between the average score of the first 5 years and the last 5 years, and difference between the earliest score the subject has and the latest score the subject has.

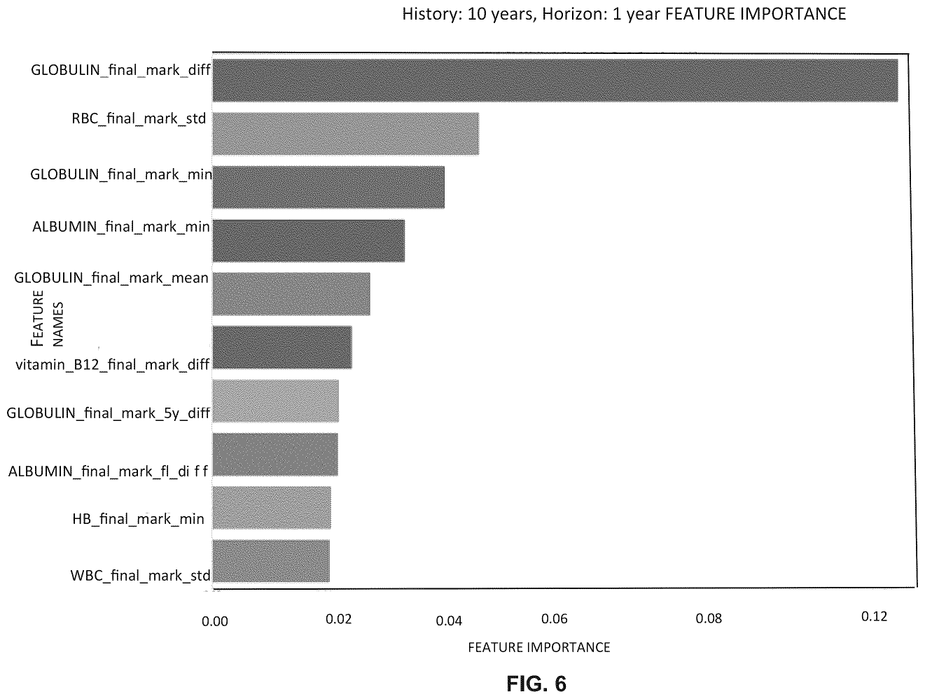

FIG. 6 shows the relative contribution or impact of each input feature (in this case, blood test values) on the predictive model's output (the predicted risk score for neurodegenerative diseases) for a one year horizon (subsequent time period) prediction based on a 10 years previous time period (history). The graph indicates that the “GLOBULIN_final_mark_diff” blood test has the highest feature importance, meaning it has the most significant impact on the predictive model's decision-making process and this suggests that changes or differences in globulin levels over time are strongly associated with the onset of neurodegenerative diseases. Other blood tests, such as “RBC_final_mark_diff”, “GLOBULIN_final_mark_min”, and “ALBUMIN_final_mark_min”, also show relatively high feature importance, indicating that they play a significant role in predicting the onset of neurodegenerative diseases. On the other hand, blood tests like “WBC_final_mark_std” and “HB_final_mark_max” have lower feature importance, suggesting that they have a less significant impact on the predictive model's output. By identifying the most important features, a subset of highly predictive blood tests can be elected, reducing the complexity and computational resources required for the predictive model.

As seen, the XGBoost model was able to correctly identify 76% of the subjects (patients) who will be diagnosed with Alzheimer's dementia one year in advance, based on data ten years before the diagnosis of the disease.

Moreover, an XGBoost model trained on five years of blood test data, was able to correctly predict which subjects will be diagnosed with Alzheimer's dementia five years in advance with sensitivity (accuracy) of 78% and precision of 81%.

Another set of experiments is now described. The additional set of experiments is different than the initial set of experiments described above, and provides support for the claims and related description of the present disclosure. For example, the new set of experiments uses a different set of blood tests than the initial set of experiments, uses a different methodology than the initial set of experiments, has a different patient selection process, uses a much larger number of subjects, performs different predictions, has improved prediction performance, and other features as described below and/or other features which are claimed in the claims and/or described in the application.

### Data

A cohort from which a training dataset was created included individuals from the Clalit (A health provider in Israel) Dan and Petach Tikva districts (geographical regions in Israel), covering Clalit-insured people living in these districts. All subjects at the age of 47 years or older as of Jan. 1, 2000 were included in the study leaving a total of 504,219 participants. Subjects who had neurodegenrative diagnosis other than Alzheimer's disease (AD) or who had other non-AD etiology, which may lead to cognitive decline, were excluded from the study (i.e. Parkinson's disease, stroke, Levy Body Demnetia, frontotoemproal dementia etc′). Those who were diagnosed with AD or who had received pharmaceutical treatments for AD—specifically, Donepezil, Galantamine, Rivastigmine, or Memantine—were defined as AD patients. AD diagnosis was based on the 2011 national institute of aging (NIA) criteria for the the diagnosis of dementia due to Alzheimer's disease. All Other subjects were referred to as cognitive healthy controls. The longitudinal cohort study spanned from 2000 to 2022, and collected data included demographic parameters (birth date, gender, socioeconomic status), recorded vital signs, smoking habits, medication, and laboratory findings. For this algorithm, we're focusing on demographic characteristics and laboratory results. Parameters like vascular risk factors were not excluded from the experiment, but it is to be understood that such parameters like vascular risk factors may be included, for example, to enhance the model's sensitivity and/or decrease its false positive rate.

### Population Distribution

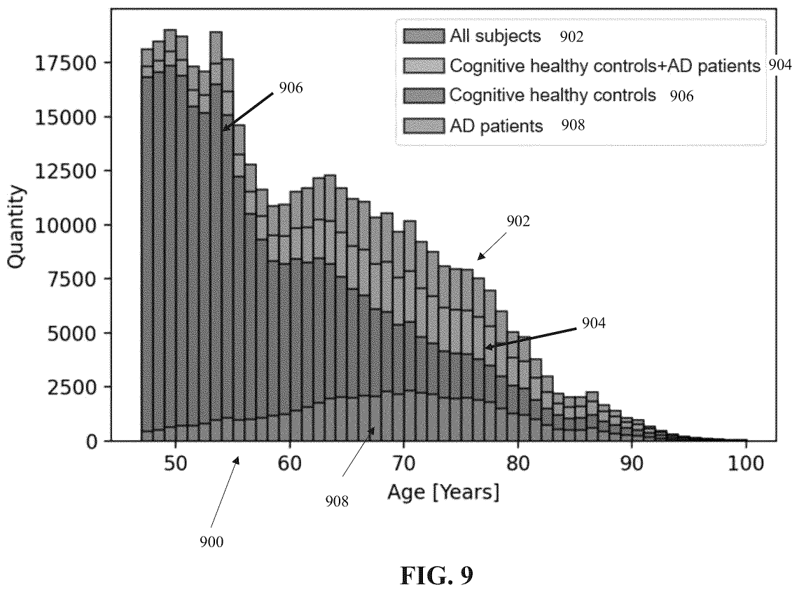

Reference is also made to FIG. 9, which is a graph 902 depicting age distribution for various classes in the population at the end of the study period (in the year 2022) of the new set of experiments, in accordance with some embodiments of the present invention. The population classes include all subjects 902, cognitive healthy controls and AD patients 904, cognitive health controls 906, and AD patients 908. The age distribution of cognitive healthy controls subjects is similar to that of the entire cohort, while the age distribution of AD patients resembles a normal distribution.

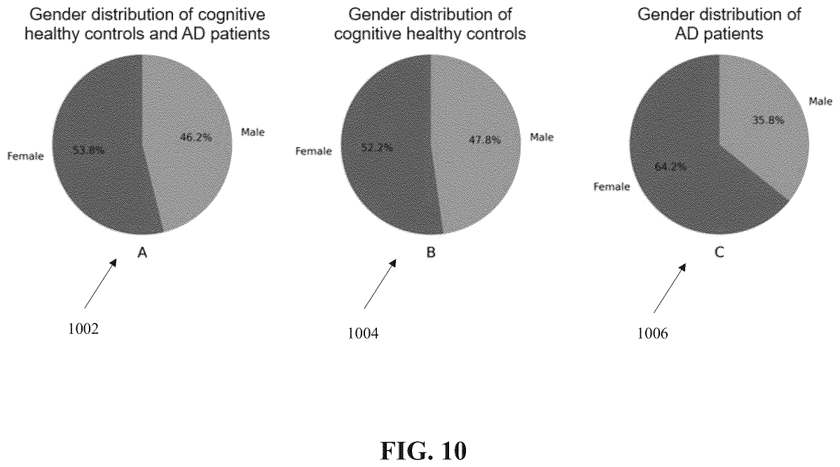

Reference is also made to FIG. 10, which includes graphs 1002, 1004, and 1006 depicting gender distribution of cognitive health controls and AD patients participating in the the new set of experiments, in accordance with some embodiments of the present invention. The gender distribution observed within the cognitive healthy controls and AD patients groups presented in graph 1002 and specifically within the cognitive healthy controls group presented in graph 1004 closely mirrors the overall gender distribution in the Israeli population. Additionally, the gender ratio (male to female) among the AD patients presented in graph 1006, which stands at 1:1.8, is consistent with the ratios documented in the medical literature.

### Data Preprocessing

The collected data was not properly tagged and was spread across multiple datasets, which presents a technical challenge. These datasets included demographic details, laboratory results, diagnoses, prescriptions and medications. To prepare the data for analysis and align it with predictive modeling frameworks, it was necessary to consolidate it into a single dataset containing all required information. Subjects were categorized into four classes based on their diagnoses and medication histories: Cognitive healthy controls, AD pateints, Cognitive decline not due to AD, and AD patients with prior non-AD diagnosis which may explain their cognitive decline (ie non-AD etiology). Cognitive decline not due to AD class were defined as those diagnosed or treated for conditions casuing cognitive decline other than AD such as Brain Tumors, Creutzfeld-Jacob Disease, Drug & Alcohol-Induced Dementia, Parkinson's disease, Lewy Body dementia, stroke, frototemproal dementia or any other neurodegenerative diagnosis. AD patients with prior non-AD diagnosis (ie non-AD etioligy) class were defined as AD pateints who had prior diagnosis which may lead to cognitive decline (same set of diagnosis as for the Cognitive decline not due to AD class). Otherwise, they were labeled as AD patients in acoordance with the 2011 NIA guidelines for the diagnosis of Alzheimer's disease dementia. Subjects not falling into these categories were assigned to Cognitive healthy controls. Out of the 504,219 subjects in our cohort, 11.8% were assigned as AD patients. This proportion is comparable to the 10.8% reported in the medical literature. For AD prediction, only the Cognitive healthy controls and AD patients sub-groups were utilized.

The subsequent preprocessing phase involved organizing the results to yield a single blood test record per patient annually. The preprocessing phases followed the common pipeline, including missing data imputation, OHE, scaling, and additional features as described herein.

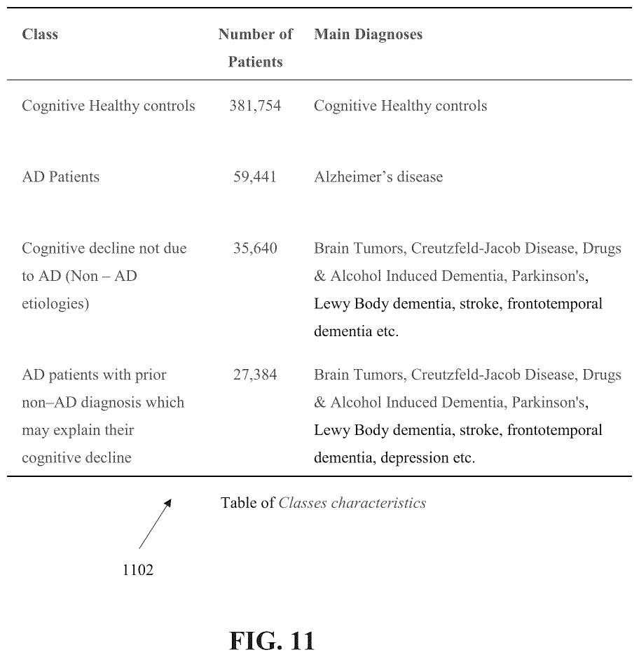

Reference is also made to FIG. 11, which is a table 1102 of characteristics of classes of subjects arranged as part of the the new set of experiments, in accordance with some embodiments of the present invention.

### Biomarkers

The processed blood test results (processed as described herein) include data from various biomarkers that have been examined out of the biomarkers available on a complete blood count (CBC) and regular blood chemistry. Two separate datasets were compiled to focus on the most frequently examined biomarkers. One dataset includes biomarkers available for over 90% of the cohort population (referred to as “Common Biomarkers” Dataset), and the other dataset consists of biomarkers accessible for approximately 50% of the cohort population (referred to as “Uncommon Biomarkers” Dataset).

### Machine Learning Models

Classification models were trained and used as described herein to forecast the probability of Alzheimer's disease (AD) diagnosis. Predictive analyses were carried out for nine different combinations of historical observation periods (1, 5, 10 years) and prediction horizons (1, 5, 10 years). For instance, a model employing 10 years of historical data to predict a diagnosis within 1 year would consider data from 2006 to 2016 for a patient diagnosed in 2017. The model training procedure involved allocating 80% of the datasets for training the classification model while reserving the remaining 20% for testing. This was done to ensure that the distributions between the training and test sets were comparable, employing the Stratified ShuffleSplit cross-validator technique, which also validated the robustness of our results. Regarding the classification process, the threshold confidence values, which determine the classification of a patient as diagnosed, were established for each model and dataset using Youden's J statistic index. Note that different threshold values may be selected to determine the confidence level needed to classify a patient as high-risk of AD. Each threshold may correspond to a specific combination of sensitivity and false positive rate. As sensitivity increases, the false positive rate will also increase and vice versa.

### Models Performance

Repeated development rounds ended with an evaluation of seven versions of the machine-learning models (as described herein) for predicting the diagnosis of AD. Two separate blood test datasets, labeled “Common Biomarkers” and “Uncommon Biomarkers,” were used for training and testing. Each model was created with one of nine combinations of historical observation periods (1, 5, 10 years) and prediction horizons (1, 5, 10 years). It should be noted that a model based on a one-year historical observation period can function with data from a single blood exam, utilizing a set of biomarkers measured at a single point in time.

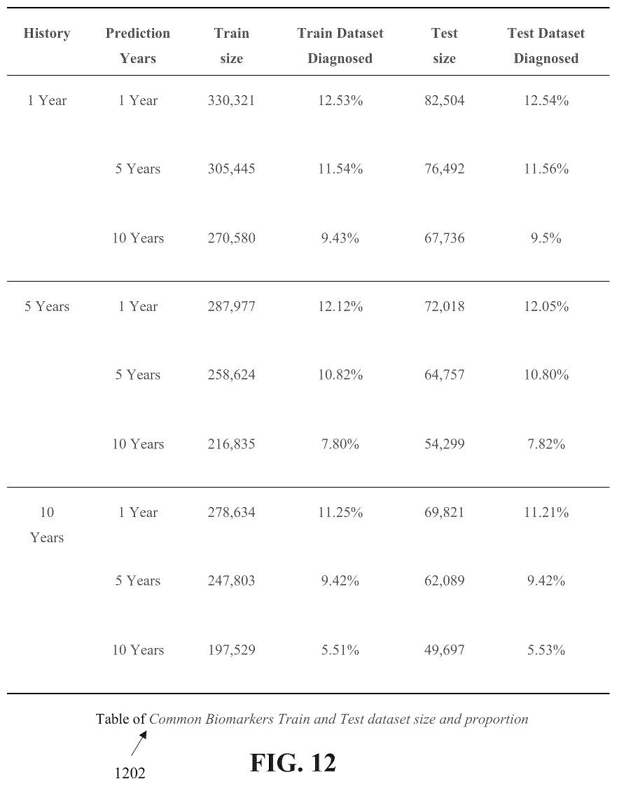

Reference is also made to FIG. 12, which is a table 1202 presenting a proportion of AD diagnosed for each train and test dataset as part of the the new set of experiments, in accordance with some embodiments of the present invention. A total of 63 models were developed for each dataset (common and uncommon biomarkers) comprising 7 models×3 history ranges×3 horizons.

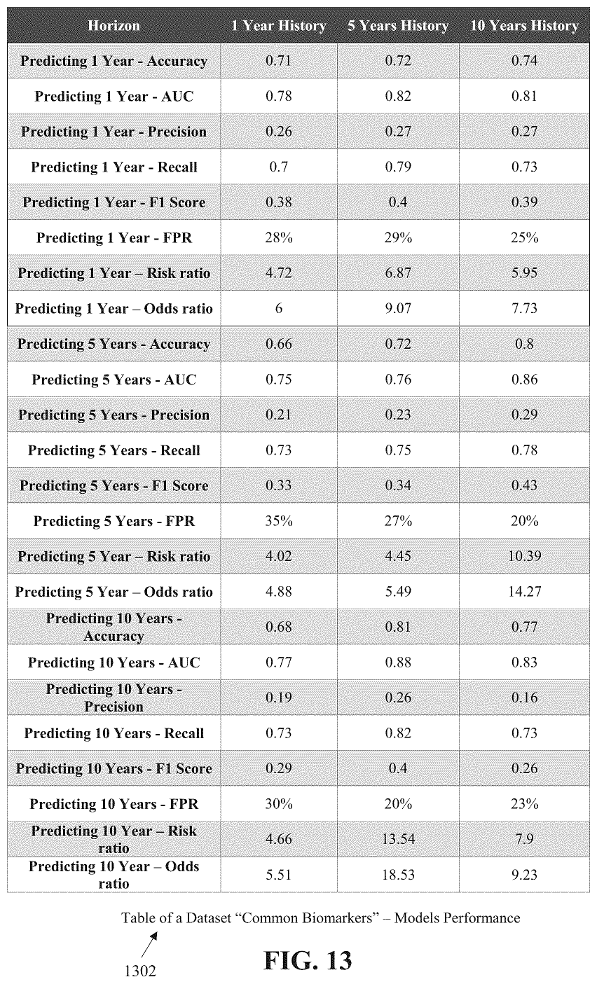

Reference is also made to FIG. 13, which is a table 1302 presenting performance metrics of the selected models for the common biomarkers of the the new set of experiments, in accordance with some embodiments of the present invention. The rows of the tables show various metrics (accuracy, AUC, precision, recall, and F1 score) for different models across 1, 5, and 10-year horizons. Notably, a model based on 5 years of historical data and a 10-year horizon stands out among the top-performing models. It achieves an accuracy of 0.81, an AUC of 0.88, a precision of 0.26, a recall of 0.82, F1 scores of 0.4, and a false positive rate of 20%.

It is expected that during the life of a patent maturing from this application many relevant systems, methods and computer programs will be developed and the scope of the term model is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was for example and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A system for predicting onset of neurodegenerative diseases, comprising:
at least one processor adapted to execute a code, the code comprising:
code instructions to receive values of a plurality of blood test measured for target subject during at least one previous time period, each plurality of blood test values is associated with a respective time stamp,
code instructions to receive at least one diagnostic test, and to verify that the at least one diagnostic test indicates non-presence of the neurodegenerative disease and/or neurodegenerative disease risk factor in the target subject during a time interval when the plurality of blood tests were measured;
code instructions to apply at least one trained predictive model to compute a predicted risk score for the target subject based on a plurality of features extracted from the plurality of blood test values, the at least one trained predictive model is trained to predict a probability of onset of at least one neurodegenerative disease and/or neurodegenerative disease risk factor in subjects during a subsequent time period based on the plurality of blood test values measured during the at least one previous time period; and
code instructions to output the predicted risk score indicative of the probability of onset of the at least one neurodegenerative disease in the target subject during the subsequent time period.

2. The system of claim 1, further comprising code instructions for excluding the plurality of blood test values from further processing when the at least one diagnostic test indicates presence of the at least one neurodegenerative disease and/or neurodegenerative disease risk factor in the target subject during a time interval when the plurality of blood tests were measured.

3. The system of claim 1, wherein at least one diagnostic test indicates a state of tau protein and/or amyloid indicative of presence or non-presence of the at least one neurodegenerative disease in the target subject.

4. The system of claim 1, further comprising code instructions for accessing an electronic health record (EMR) or lab results database of the subject, and verifying at least one of:
(1) lack of symptoms correlated with the at least one neurodegenerative disease during the time interval when the plurality of blood tests were measured, or excluding the plurality of blood test values from further processing when the EMR includes symptoms correlated with the at least one neurodegenerative disease, or not having neurodegenerative disease risk factor, and
(2) lack of administered medications for treatment of the at least one neurodegenerative disease.

5. The system of claim 1, wherein the result of the at least one diagnostic test is non-correlated with a stage of the at least one neurodegenerative disease on a predefined clinical scale of a plurality of stages for diagnosing the at least one neurodegenerative disease.

6. The system of claim 1, wherein the at least one trained predictive model comprises at least one first trained predictive model, wherein the plurality of features comprises a first plurality of features, wherein the predicted risk score comprises a first predicted risk score, wherein the subsequent time period comprises a first subsequent time period,
the code further comprises code instructions to apply at least one second trained predictive model to compute a second predicted risk score for the target subject based on a second plurality of features extracted from the plurality of blood test values and/or based on the plurality of blood test values, the at least one second trained predictive model is trained to predict a second probability of onset of at least one risk factor likely to lead to development of the at least one neurodegenerative disease in the target subject during a second subsequent time period prior to the first subsequent time period predicted for onset of the at least one neurodegenerative disease.

7. The system of claim 6, wherein the at least one second trained predictive model is applied in response to the first predicted risk score being above a threshold, wherein the second predicted risk score indicates probability of onset of the at least one risk factor in view of the first predicted risk score being above the threshold.

8. The system of claim 6, wherein the at least one risk factor is determined by at least one of the plurality of blood test values are within a target range, wherein the plurality of blood test values are external to the target range during the time interval when the plurality of blood tests were measured.

9. The system of claim 6, further comprising code for applying an interpretability model to the at least one first trained model to identify at least one first blood test value correlated with the first predicted risk score above a first threshold, and applying the interpretability model to the at least one second trained model to identify at least one second blood test value correlated with the second predicted risk score above a second threshold, and confirming that the at least one first blood test value matches the at least one second blood test value.

10. The system of claim 6, wherein the at least one second trained predictive model is trained on a training dataset of a plurality of records, wherein a record is for a sample individual, the record including the first blood test values and/or the plurality of extracted features, and a ground truth indicating onset or non-onset of at least one risk factor, and onset or non-onset of the at least one neurodegenerative disease, wherein a plurality of records are for sample individuals with a first time interval of onset of the at least one risk factor followed by a second subsequent time interval with onset of the at least one neurodegenerative disease.

11. The system of claim 6, in response to the second predicted risk score indicating the at least one risk factor being above a threshold, treating the target subject for preventing onset of the at least one risk factor.

12. The system of claim 1, further comprising: in response to the predicted risk score being above a threshold, monitoring the EMR or blood results database of the target subject during the subsequent time period to identify at least one of: an administered cognitive evaluation indicative cognitive decline, and/or the at least one diagnostic test indicating presence of the at least one neurodegenerative disease in the target subject, and treating the target subject by administering at least one medication to the target subject effective for delaying or preventing progression of the at least one neurodegenerative disease.

13. The system of claim 12, wherein the at least one neurodegenerative disease includes Alzheimer's Disease, and at least one medication is selected from drugs against the amyloid protein selected from LEQEMBI, Donanemab, aducanumab, or drugs against the tau protein including Tau-targeted therapies, and drugs aimed at the immune system, selected from IBC-ABb002.

14. The system of claim 1, further comprising: in response to the predicted risk score being above a threshold, treating the target subject by administering at least one medication known to be effective for preventing onset of the neurodegenerative disease.

15. The system of claim 1, further comprising in response to the predicted risk score being above a threshold, monitoring the EMR of the target subject during the subsequent time period to identify at least one of: an administered cognitive evaluation indicative of normal cognitive function and/or lack of cognitive decline, and the at least one diagnostic test indicating presence of the at least one neurodegenerative disease in the target subject, and treating the target subject by administering at least one medication to the target subject effective for preventing onset of clinical appearance of the at least one neurodegenerative disease.

16. The system of claim 15, wherein the at least one neurodegenerative disease includes Alzheimer's Disease, and at least one medication is selected from LEQEMBI, and Donanemab.

17. The system of claim 1, wherein the at least one trained predictive model generates a respective predicted risk score for each stage of a plurality of sequential stages denoting a disease progression profile defined according to a clinical standard.

18. The system of claim 17, wherein the at least one trained predicted mode is applied to a temporal sequence of a plurality of sets of the plurality of blood test values, each respective set obtained at a respective historical time interval along the temporal sequence.

19. The system of claim 17, wherein the at least one trained predictive model is trained on a training dataset of a plurality of records, wherein a record is for a sample individual, wherein the record includes a plurality of sets of the plurality of blood test values and/or the plurality of features for the sample individual, a timestamp indicating date for each set of the plurality of sets, and a ground truth indicating diagnosis of at least one stage of the plurality of stages, and a timestamp indicating date for each stage.

20. The system of claim 1, further comprising code for creating a training dataset, comprising:
accessing a plurality of EMRs for a plurality of sample individuals;
analyzing each EMR of the plurality of EMRs for creating a subset of EMRs by:
including EMRs of sample individuals diagnosed with Alzheimer's Disease (AD);
excluding EMRs of sample individuals diagnosed with neurodegenerative diseases other than AD, or who had other non-AD etiology, correlated with cognitive decline; and
including other EMRs of sample individuals not diagnosed with AD and not excluded, as cognitive healthy controls;
for each EMR of the subset of EMRs, generating a record including at least one set of the plurality of blood test values, a first timestamp indicating date of the set, and a ground truth indicating AD or cognitive healthy control, and a second timestamp indicating date of diagnosis of AD.

21. The system of claim 20, wherein EMRs of individuals diagnosed with any one of the following conditions leading to cognitive decline document in the EMR are excluded: Brain Tumors, Creutzfeld-Jacob Disease, Drug & Alcohol-Induced Dementia, Parkinson's disease, Lewy Body dementia, stroke, frototemproal dementia.

22. The system of claim 20, wherein EMRs of sample individuals diagnosed with AD are identified according to at least one of: a diagnostic field indicating AD, and indication of prescribed pharmaceutical treatments known to be prescribed for AD.

23. The system of claim 20, further comprising code for analyzing the EMRs of the sample individuals, and classifying each EMR of each sample individual into a classification category selected from: Cognitive healthy controls, AD pateints, Cognitive decline not due to AD, and AD patients with prior non-AD diagnosis which is correlated with cognitive decline.

24. A method for training a predictive model to predict the onset of neurodegenerative diseases, comprising:
receiving blood test values for a plurality of subjects over a previous time period, wherein each blood test value is associated with a timestamp and each target subject is associated with a label indicating the presence or absence of a neurodegenerative disease;
wherein the label is determined for each subject by receiving at least one diagnostic test, and assigning the label indicating absence when the at least one diagnostic test indicates non-presence of the clinical stage of the neurodegenerative disease in the subject during a time interval when the blood test values were measured, and assigning the label indicating presence when the at least one diagnostic test indicates presence of the neurodegenerative disease;
extracting a plurality of features from the blood test values, including at least one of:
an aggregation of blood test values over the previous time period, selected from the group consisting of an average value, a maximum value, a minimum value, and a standard deviation;
a change pattern in the blood test values over the previous time period, selected from the group consisting of values increasing over time, values decreasing over time, values increasing then decreasing, and
significant alternations between increases and decreases;
training a predictive model using the extracted features and the labels to predict the onset of neurodegenerative diseases; and
outputting the trained predictive model for classification the onset of one or more neurodegenerative diseases of a target.
